SEQUENCE LISTING (33 sequences)

SEQ ID NO: 1 - DNA, 36179 bp, Unknown
  Coronaviridae
  misc_feature: Nucleotide sequence of the vector containing the complete pBAC-MERS-CoV-[3,4a,4b,5,E] replicon. Includes pBAC sequence (nucleotides 1 to 7889), RNA replicon (nucleotides 7890 to 35838) and pBAC sequence (nucleotides 35839 to 36179)
agcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtagacctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgcctatggcgcgcctaagttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtgatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtacttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaatttagtgtgaatctttttgacgttggttactcagttaattagtcccgtgtagaggctaatccattagtctctctttggacatatggtgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaaaaaaaaaaaaaaagggtcggcatggcatctccacctcctcgcggtccgacctgggcatccgaaggaggacgtcgtccactcggatggctaagggagagctcggatcgatccgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcgagctctgtacatgtccgcgga

SEQ ID NO: 2 - DNA, 36173 bp, Unknown
  Coronaviridae
  misc_feature: Nucleotide sequence of the vector containing the full-length pBAC-MERS-MA30-?[3,4a,4b,5,E] replicon. Includes pBAC sequence (nucleotides 1 to 7889), RNA replicon (nucleotides 7890 to 35838) and pBAC sequence (nucleotides 35832 to 36173)
agcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtagacctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgcctatggcgcgcctaagttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtgatttaagtgaatagcttggctatctcgcttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtttacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtttgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtgttggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaactgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagcgagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtacttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtgatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgccgtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaatagtgtgaatctttttgacgttggttactcagttagtcccgtgtagaggctaatccattagtctctctttggacatatggtgtaaaattccaggatagtaaaccccctctaccacctgacgagtgggtttaacgaactccttcataatgtctattatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcgtctgtcgtagtcacaagcaccttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaaaccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaagtagatgatttgcaaaaaaaaaaaaaaaaaaaaaaaaagggtcggcatggcatctccacctcctcgcggtccgacctgggcatccgaaggaggacgtcgtccactcggatggctaagggagagctcggatcgatccgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcgagctctgtacatgtccgcgga

SEQ ID NO: 3 - DNA, 4062 bp, Unknown
  Coronaviridae
  gene (1)..(4062): Nucleotide sequence of the gene encoding the S protein of MERS-MA30-CoV with codons optimised for expression in mammalian cells
atgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagagacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagagtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactggatgtgctggagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactga

SEQ ID NO: 4 - DNA, 4062 bp, Unknown
  Coronaviridae
  gene (1)..(4062): Nucleotide sequence of the gene encoding MERS-MA30-CoV S protein with codons not optimised for expression in mammalian cells
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtgatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgccgtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaa

SEQ ID NO: 5 - DNA, 4062 bp, Unknown
  Coronaviridae
  gene (1)..(4062): Nucleotide sequence of the gene encoding MERS-CoV protein S with codons optimised for expression in mammalian cells
atgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagaaacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagcgtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactggatgtgctggagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactga

SEQ ID NO: 6 - DNA, 4062 bp, Unknown
  Coronaviridae
  GENE (1)..(4062): Nucleotide sequence of the gene encoding MERS-CoV Protein S with codons not optimised for expression in mammalian cells
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaa

SEQ ID NO: 7 - DNA, 4062 bp, Unknown
  Coronaviridae
  GENE (1)..(4062): Nucleotide sequence of the gene encoding MERS-MA30-CoV protein S with codons optimised for expression in mammalian cells and modifications 24633_24634 delins CC and 24637_24638 delins CC
atgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagagacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagagtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactcgatccgcccgagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactga

SEQ ID NO: 8 - DNA, 4062 bp, Unknown
  Coronaviridae
  GENE (1)..(4062): Nucleotide sequence of the gene encoding the S protein of MERS-CoV with codons optimised for expression in mammalian cells and modifications 24633_24634 delins CC and 24637_24638 delins CC
atgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagaaacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagcgtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactcgatccgcccgagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactga

SEQ ID NO: 9 - DNA, 27924 bp, Unknown
  Coronaviridae
  misc_feature: Nucleotide sequence of the chemically defined MERS-MA30-V1-CD replicon
gatttaagtgaatagcttggctatctcgcttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatgcagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtttacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtttgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtgttggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaactgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagcgagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtacttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagagacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagagtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactcgatccgcccgagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactgatagtgtgaatctttttgacgttggttactcagttagtcccgtgtagaggctaatccattagtctctctttggacatatggtgtaaaattccaggatagtaaaccccctctaccacctgacgagtgggtttaacgaactccttcataatgtctattatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcgtctgtcgtagtcacaagcaccttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaaaccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaagtagatgatttgcaaaaaaaaaaaaaaaaaaaaaaaaaaaaaaaaaaaaaaaaaa

SEQ ID NO: 10 - DNA, 27924 bp, Unknown
  Coronaviridae
  misc_feature: Nucleotide sequence of the chemically defined MERS-CoV-V1-CD replicon
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatgcagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtacttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagaaacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagcgtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactcgatccgcccgagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactgatagtgtgaatctttttgacgttggttactcagttagtcccgtgtagaggctaatccattagtctctctttggacatatggtgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaaaaaaaaaaaaaaaaaaaaaaaaaaaaaaaa

SEQ ID NO: 11 - DNA, 27907 bp, Unknown
  Coronaviridae
  misc_feature: Nucleotide sequence of the MERS-MA30-V1-VLP replicon
gatttaagtgaatagcttggctatctcgcttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatgcagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtttacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtttgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtgttggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaactgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagcgagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtacttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagagacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagagtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactggatgtgctggagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactgatagtgtgaatctttttgacgttggttactcagttagtcccgtgtagaggctaatccattagtctctctttggacatatggtgtaaaattccaggatagtaaaccccctctaccacctgacgagtgggtttaacgaactccttcataatgtctattatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcgtctgtcgtagtcacaagcaccttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaaaccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaagtagatgatttgcaaaaaaaaaaaaaaaaaaaaaaaaa

SEQ ID NO: 12 - DNA, 27907 bp, Unknown
  Coronaviridae
  misc_feature: Nucleotide sequence of the MERS-CoV-V1-VLP replicon
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatgcagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtacttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgattcacagcgtgttcctgctgatgtttctgctgacacccacggagtcctacgtggacgtgggaccagacagcgtgaagtcagcctgcatcgaagtggacatccagcagacatttttcgataagacctggcctcggcccattgatgtgagcaaggccgacggcatcatctatcctcagggcagaacatacagcaatatcaccatcacctatcagggcctgttcccttaccagggcgatcatggagacatgtacgtgtacagcgccggccatgctaccgggaccacccctcagaaactgttcgtggcaaactactcacaggacgtgaagcagttcgctaacgggtttgtggtgagaatcggagccgccgccaactccaccgggaccgtgatcatcagcccatcaacaagtgccaccatcaggaagatttaccctgccttcatgctggggtccagcgtcggcaacttcagcgatggcaagatgggccggtttttcaaccataccctggtgctgctgccagacggctgcggaaccctgctgagagccttctactgcatccttgagccacgcagcggcaatcactgtcccgccggaaattcctacacatcctttgccacatatcacactcccgcaacagactgctctgacggtaattataatagaaacgccagcctgaacagctttaaagagtactttaacctgaggaactgtacttttatgtatacctacaacattaccgaagacgagatcctggaatggttcggtatcactcagactgcccagggggtccacctgtttagcagcagatacgtggatctgtacggcggcaacatgttccagtttgccaccctgccagtctacgacaccattaagtactacagtattatcccccactccatccggtctatccagtctgaccggaaggcttgggccgccttttatgtgtataagctgcagcccctcacctttctgctggactttagtgtggacggctacatcagacgcgccatcgactgtggcttcaacgatctgtcccagctgcactgcagctacgagtctttcgatgtggagtctggcgtgtatagcgttagtagctttgaggccaaacctagcgggtccgtggtggagcaggccgagggggtggagtgcgattttagccctctgctgagcggcacccctcctcaggtgtataacttcaagagactggtgttcaccaattgcaactataacctgacaaagctgctctcactcttctccgttaacgacttcacctgcagccagatctcacctgccgctatcgccagcaattgctattcctctctgattctggattactttagttatcctctctcaatgaagtccgacctttccgtgagctccgccggccctatctcccagttcaattacaaacagagttttagcaaccctacatgcctgattctggccaccgtcccccacaatctgaccaccatcaccaagcccctgaagtatagctatattaacaagtgctccaggctgctgagcgatgacaggaccgaggtgccacaactggtgaatgccaatcagtactccccttgcgtgagcattgtgccaagcaccgtctgggaagacggagactactatcggaagcagctgtcccccctggagggcggaggctggctggtggcttccggctctaccgtggccatgaccgagcagctgcagatggggttcggcatcacagtgcagtacggaaccgacacaaactccgtgtgccctaagctggaatttgccaatgatactaagattgccagccagctgggcaactgcgtggagtactccctgtacggcgtgtctggccggggggtgttccagaactgcaccgccgtgggggtgaggcagcagcgcttcgtgtatgacgcctaccagaacctggtgggttattacagcgacgacggtaactactattgcctgcgggcttgtgtgagtgtgccagtgtccgtgatttacgacaaagagaccaaaacccacgccaccctgtttggcagcgtggcttgtgagcacatcagctccaccatgagccagtattccaggtctacccgctccatgctgaagcggagagactccacctacgggcccctgcagactcccgtggggtgtgttctgggcctggtgaactcctccctgtttgtggaggattgcaagctgcccctgggccagtccctgtgcgcactgcctgatacccccagcaccctgacccctagaagcgtgcgcagcgtgcctggggagatgaggctggcctcgatcgcctttaatcatcctatccaggtggaccagctgaacagctcctatttcaagctgtccatccccactaacttctcatttggcgtgacccaggagtatatccagactaccatccagaaggtgactgtggattgtaagcagtacgtgtgcaatggcttccagaagtgcgaacagctgctgagagagtacggccagttttgctcaaagatcaaccaggctctgcatggagccaatctgaggcaggatgactctgtgcgcaacctgttcgcatccgtgaaatccagccagagctctcccatcattcctgggttcgggggcgactttaacctgaccctgctcgagcctgtctccattagcactggctccaggtccgccagaagtgccattgaggatctgctcttcgacaaagtgacaattgctgatccaggatatatgcaaggatacgacgactgcatgcagcaggggcccgcttccgcaagagacctgatctgcgcccagtacgttgccggctacaaggtgctgccacccctgatggacgtgaacatggaggccgcctacacctcttccctgctcggcagcatcgctggcgtggggtggaccgccgggctgagctccttcgccgctatcccttttgcacagtccatcttttaccggctgaacggggtggggatcacacagcaggtgctgagcgagaaccagaagctcatcgccaacaaattcaaccaggccctgggcgccatgcagactggctttacaactactaatgaagccttccagaaggtgcaggacgccgtgaataacaacgcccaggccctgtctaaattggccagtgagctgtctaatacttttggggccatctccgcatctatcggcgatatcattcaaagactggatgtgctggagcaggacgcccagattgaccgcctgatcaatgggagactgaccactcttaatgccttcgtggctcagcagctggtgagaagcgagagcgctgctctgtccgcacagctggcaaaggacaaagtgaacgagtgtgtgaaagctcagtcaaagaggagcgggttttgtggccagggaactcacattgtgagctttgtggtgaacgctcccaacggcctgtactttatgcatgtgggctattacccatccaatcatatcgaagtggtgtctgcctatggactgtgcgacgccgctaatccaaccaactgcatcgcacctgtgaatgggtactttattaaaacaaataatacccggatcgtcgacgaatggtcctacaccggcagcagcttttacgcccctgagcccattacatccctgaacacaaagtatgtcgccccacaggtgacataccagaacatcagcaccaacttgccaccacctctgctggggaatagcaccggcatcgactttcaggacgagctggatgagttcttcaagaatgtgagcacatccatcccaaacttcggcagcctgacccagatcaacacaaccctgcttgacctgacttatgaaatgctgagcctgcagcaggtggttaaggcgctgaatgaatcttacatcgatctgaaggaactgggcaactatacctactataacaaatggccctggtatatctggctggggtttattgccggcctggtggctctggctctgtgcgtgtttttcatcctgtgctgcaccggatgcggaacaaattgcatgggcaagctgaaatgcaatcggtgctgcgacaggtatgaggagtacgatctggagccacacaaggtgcatgtgcactgatagtgtgaatctttttgacgttggttactcagttagtcccgtgtagaggctaatccattagtctctctttggacatatggtgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaaaaaaaaaaaaaaa

SEQ ID NO: 13 - DNA, 18 bp, Artificial Sequence
  PART OF A VECTOR (promotor T7P)
  promoter (1)..(18): Nucleotide sequence of the T7P promoter
taatacgactcactatag

SEQ ID NO: 14 - DNA, 44 bp, Artificial Sequence
  vector part
  5′UTR (1)..(44): Nucleotide sequence of the 5′UTR
ataggcggcgcatgagagaagcccagaccaattacctacccaaa

SEQ ID NO: 15 - DNA, 195 bp, Venezuelan equine encephalitis virus
  misc_feature: MISC nucleotide sequence
taggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattgga

SEQ ID NO: 16 - DNA, 142 bp, Sindbis virus
  misc_feature: DLP nucleotide sequence
atagtcagcatagtacatttcatctgactaatactacaacaccaccaccatgaatagaggattctttaacatgctcggccgccgccccttcccggcccccactgccatgtggaggccgcggagaaggaggcaggcggccccg

SEQ ID NO: 17 - DNA, 66 bp, Porcine Teschovirus-1
  misc_feature: P2A nucleotide sequence
ggaagcggagctactaacttcagcctgctgaagcaggctggagacgtggaggagaaccctggacct

SEQ ID NO: 18 - DNA, 117 bp, Artificial Sequence
  PART OF A VECTOR
  3′UTR (1)..(117): 3′UTR nucleotide sequence
atacagcagcaattggcaagctgcttacatagaactcgcggcgattggcatgccgctttaaaatttttattttatttttcttttcttttccgaatcggattttgtttttaatatttc

SEQ ID NO: 19 - DNA, 40 bp, Artificial Sequence
  PART OF A VECTOR
  polyA_signal (1)..(40): PolyA tail nucleotide sequence
aaaaaaaaaaaaaaaaaaaaaaaaaaaaaaaaaaaaaaaa

SEQ ID NO: 20 - DNA, 27 bp, Artificial Sequence
  PART OF A VECTOR
  terminator (1)..(27): T7 terminator nucleotide sequence
cccctctctaaacggaggggttttttt

SEQ ID NO: 21 - DNA, 30115 bp, Unknown
  Coronaviridae
  misc_feature: Nucleotide sequence of MERS-MA30 virus
gatttaagtgaatagcttggctatctcgcttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtttacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtttgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtgttggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaactgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagcgagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtacttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtgatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgccgtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaagctcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttagtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctattatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaaaccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaagtagatgatttgcaaaaaaaaaaaaaaaaaaaaaaaaa

SEQ ID NO: 22 - DNA, 63 bp, Artificial Sequence
  PRIMER
  primer_bind (1)..(63): Primer nucleotide sequence (PCR primer) of VS-EcoRI-E-MERS-rtTA-V10-2T
ccggaattcgagctcggtacccggggatccaccggtcgccaccatgttaccctttgtccaaga

SEQ ID NO: 23 - DNA, 44 bp, Artificial Sequence
  PRIMER
  primer_bind (1)..(44): Primer nucleotide sequence (PCR primer) of RS-EcoRI-EMERS-rtTA-V10-2T
ccggaattcatgcggccgcgactctattaaacccactcgtcagg

SEQ ID NO: 24 - DNA, 18 bp, Artificial Sequence
  PRIMER
  primer_bind (1)..(18): Primer nucleotide sequence (PCR primer) of RS-E-MERS
ttaaacccactcgtcagg

SEQ ID NO: 25 - DNA, 20 bp, Artificial Sequence
  PRIMER
  primer_bind (1)..(20): Primer nucleotide sequence (PCR primer) of VS-TRE-Auto-2380
atccacgctgttttgacctc

SEQ ID NO: 26 - DNA, 17 bp, Artificial Sequence
  PRIMER
  primer_bind (1)..(17): Primer nucleotide sequence (Taqman probes) gRNA-MERS
tgctccaacagttacac

SEQ ID NO: 27 - DNA, 18 bp, Artificial Sequence
  PRIMER
  primer_bind (1)..(18): Primer nucleotide sequence (Taqman probes) sgmRNA-N-MERS
ctttgattttaacgaatc

SEQ ID NO: 28 - DNA, 23 bp, Artificial Sequence
  PRIMER
  primer_bind (1)..(23): Primer nucleotide sequence (PCR primer) of VS MERS gRNA
gcacatctgtggttctcctctct

SEQ ID NO: 29 - DNA, 22 bp, Artificial Sequence
  PRIMER
  primer_bind (1)..(22): Primer nucleotide sequence (PCR primer) of RS MERS gRNA
aagcccaggccctactattagc

SEQ ID NO: 30 - DNA, 20 bp, Artificial Sequence
  PRIMER
  primer_bind (1)..(20): Primer nucleotide sequence (PCR primer) of Leader sgRNA
cttcccctcgttctcttgca

SEQ ID NO: 31 - DNA, 22 bp, Artificial Sequence
  PRIMER
  primer_bind (1)..(22): Primer nucleotide sequence (PCR primer) of sgRNA-N
tcattgttatcggcaaaggaaa

SEQ ID NO: 32 - protein, 8 aa, Artificial Sequence
  Cyclic peptide
QAEAQAEA

SEQ ID NO: 33 - protein, 12 aa, Artificial Sequence
  TAT dodecapeptide that facilitate cell penetration
GRKKRRQRRRPQ

(19) United States
(12) Patent Application Publication
(10) Pub. No.: US 2023/0203536 A1
(43) Pub. Date: Jun. 29, 2023

(54) CORONAVIRUS RNA REPLICONS AND USE THEREOF AS VACCINES

(71) Applicant: CONSEJO SUPERIOR DE INVESTIGACIONES CIENTÍFICAS (CSIC), Madrid (ES)

(72) Inventors: Luis ENJUANES SÁNCHEZ, Madrid (ES); María Isabel SOLA GURPEGUI, Madrid (ES); Sonia ZÚÑIGA LUCAS, Madrid (ES); Francisco Javier GUTIÉRREZ ÁLVAREZ, Madrid (ES); José Manuel HONRUBIA BELENGUER, Madrid (ES); Raúl FERNÁNDEZ DELGADO, Madrid (ES)

(21) Appl. No.: 17/928,183

(22) PCT Filed: May 26, 2021
(86) PCT No.: PCT/ES2021/070378

§ 371 Date: Nov. 28, 2022

(30) Foreign Application Priority Data
May 28, 2020 (ES) P202030498

(51) Int. Cl. C12N 15/86; A61K 39/215; A61P 31/14

(52) U.S. Cl. CPC C12N 15/86; A61K 39/215; A61P 31/14; A61K 2039/5254

(57) ABSTRACT

RNA replicon derived from a coronavirus with complete or partial deletion of: the gene encoding the E protein and at least 4 genes encoding genus accessory proteins selected from: 3, 4a, 4b and 5, in the case of MERS-CoV. Method of preparation thereof, and their use in vaccine compositions.

### FIELD OF THE INVENTION

The invention falls within the field of recombinant genetic engineering. An RNA replicon deficient in propagation, obtained from a coronavirus is described, as well as the method for its generation. This method comprises the total or partial deletion of at least 5 genes of the coronavirus: a gene that encodes protein E and at least 4 genes that encode genus-specific accessory proteins. These RNA replicons have been derived from the MERS-CoV genome. The composition comprising said RNA replicon for use as a vaccine, to generate immunity against coronavirus infection, is described.

### BACKGROUND OF THE INVENTION

Coronaviruses (CoVs) are a family of positive-sense single-stranded RNA (ssRNA+) viruses that have the largest known genome for an RNA virus, with a length between approximately 25 and 33 kilobases (kb). During coronavirus infection, genomic RNA (gRNA) replication occurs and a set of subgenomic RNAs (sgRNA) of positive and negative polarity are synthesized without using reverse transcription.

Coronaviruses mainly cause infections in birds and mammals, including humans. The severe acute respiratory syndrome coronavirus (SARS-CoV) outbreak in 2002 and, more recently, the Middle East respiratory syndrome coronavirus (MERS-CoV) in 2012, have demonstrated the lethality of CoVs when they cross the species barrier and infect humans. For this reason, vaccines against these pathogens are necessary and one option is RNA replicon-based vaccines development.

Propagation-deficient RNA replicons are excellent platforms for the generation of vaccines, since they are a subtype of vaccines derived from viruses, with a single infective cycle as they cannot spread from cell to cell. The deficiency of these replicons in one or more essential functions for the synthesis, assembly of viral particles or dissemination, make them very safe vaccines and highly useful as vectors for immunization against infectious agents (Lundstrom, 2018). To amplify these replicons, it is convenient to provide in trans the viral genes necessary for their propagation, which have been previously deleted from them. To this end, replicons can be grown in cell lines that complement and express the required viral proteins missing in them, required for their dissemination (Almazán et al., 2013; Ortego et al., 2002). When replicons grow in cells that do not complement their deficiencies, for example, inside the subject that has been vaccinated with the RNA replicon, they express their deficient genomes and the antigens they encode, without being able to produce infective virions able to disseminate from cell to cell.

RNA replicons can be classified into two main groups: (i) replication-defective and (ii) replication-competent but propagation-defective. The present invention describes replication-competent, but propagation-defective RNA replicons. Some of the advantages of using RNA replicons as platforms for the generation of vaccines are: (i) their easy administration, (ii) they only have one cycle of infection due to the deleted genes, (iii) they do not integrate in the genome since they are RNAs, and (iv) they are highly biosafe.

The arrangement of the genes in the genome of a coronavirus is: 5′-UTR end (untranslated region)—replicase/transcriptase—spike (S) protein—envelope (E) protein—membrane (M) protein—nucleocapsid (N) protein—3′ UTR end and poly(A) tail. All four structural proteins (S, E, M, and N) contribute to the efficient formation of structurally stable viral particles.

In addition to structural genes, the genome of coronaviruses also contains genes that code for proteins with non-structural functions, for example, RNA replicase/transcriptase. Other genes that do not code for structural proteins are in the genome after the replicase/transcriptase gene. They are named genus-specific or genus-accessory genes. Some of these coronavirus proteins are involved in counteracting host defenses. The genes of coronaviruses are called ORF (Open Reading Frame) followed by a number. The following tables describe the distribution of genes in the MERS-CoV genome (Table 1).

TABLE 1
Genes present and their distribution in the MERS-CoV genome.

| ORF | PROTEIN | START (nt) | END (nt) |
|---|---|---|---|
| 1ab | pp1ab | 279 | 21514 |
| 2 | S | 21456 | 25517 |
| 3 | 3 | 25532 | 25843 |
| 4a | 4a | 25852 | 26181 |
| 4b | 4b | 26093 | 26833 |
| 5 | 5 | 26840 | 27514 |
| 6 | E | 27590 | 27838 |
| 7 | M | 27853 | 28512 |
| 8 | N | 28566 | 29807 |
| 8b | 8b | 28762 | 29100 |

WO2018160977 discloses an attenuated coronavirus by an alteration in the replicase gene. The present invention represents an improvement since, by keeping intact this gene, numerous antigens are produced, thus increasing the efficacy of the vaccine.

In the scientific article by Almazán et al 2013, an RNA replicon obtained from the MERS-CoV coronavirus is disclosed, in which the gene encoding the E protein has been deleted. The present invention represents an improvement over this article as the deletion of the gene encoding the E protein together with the deletion of other genus-specific accessory genes of MERS-CoV achieves a higher attenuation of the virus, and a greater safety dependent on the deletion of several genes, than the one achieved exclusively with the deletion of the gene that encodes protein E.

The present invention describes coronavirus RNA replicons, as well as the method to obtain them, and their use as vaccines. The inventors have demonstrated the attenuation and efficacy of several MERS-CoV-based coronavirus replicons in the protection against infection by human pathogenic coronaviruses. These replicons are replication-competent but propagation-defective and confer immunity against the coronaviruses from which they are derived.

### BRIEF DESCRIPTION OF THE INVENTION

The present invention relates to a self-replicating but propagating defective RNA, that is, an RNA replicon. Said replicon can be used as a vaccine composition for coronaviruses, preferably for MERS-CoV.

To achieve an RNA replicon it is necessary to delete genes that are involved in propagation, but not in replication. Among the structural genes of coronaviruses, the most relevant to be deleted, in order to obtain propagation-deficient replicons, is the gene that encodes the E protein. The present invention combines the deletion of this gene with the deletion of other genes, called genus-specific accessory genes not essential for RNA replication. Within this category are those genes encoding genus-specific accessory proteins, which may also be involved in the virulence. The main advantage of excising the gene that codes for the E protein and at least 4 genes (3, 4a, 4b, and 5) is that an increase in the safety of the RNA replicon is achieved, as the probability of reversing the five modifications recovering virulence is very low.

An additional advantage of these RNA replicons is that by keeping the replication capacity intact, when the vaccine is inoculated into a subject, the replicon will begin to replicate inside the cell, but the new RNAs and proteins encoded by genes that have not been deleted will be able to form Virus-like particles (VLPs) that will protect the RNA that forms the replicon genome against degradation. Nevertheless, these VLPs will not be able to disseminate to other cells. The viral proteins synthesized by the cell infected by the RNA replicon will form VLPs with highly immunogenic polymeric structures that will be recognized as antigens by the immune system, eliciting high and long-lasting immune responses, i.e., long immunological memory.

These RNA replicons can be used as vaccines to immunize subjects, to prevent the development of the disease caused by the coronavirus from which the replicon has been derived. Since the genes encoding the structural proteins normally recognized by the immune system have not been deleted, the immunogenic capacity of the VLPs formed by these replicons is very high. However, the VLPs produced by the replicons are defective in propagation and do not leave the cell unless the cell membrane is desintegrated.

The main advantages and novelties of the present invention are listed below:
It is the first time that a vaccine has been obtained from an RNA replicon of a coronavirus in which at least five genes have been deleted in the same coronavirus. The deletion of at least three of these proteins, transform the original virus into an attenuated RNA, competent in replication, but defective in propagation. Therefore, the engineered RNA cannot be considered a virus any more.
The strategy followed to obtain these replicons leaves the replication machinery intact, which makes possible to amplify the number of proteins that can be expressed to prime the immune system.
RNA replicon activity can also be reconstituted from various expression vectors including the required combination of genes. This provides greater biosafety during production.
The VLPs in which the RNA replicons are wrapped are indistinguishable under the electron microscope from the particles of a complete coronavirus, and the administration of these vaccines to a subject through the nasal route allows to mimic the infection route of the native virus.
Additionally, RNA replicons can be administered in combination with a polymer, e.g., a cationic polymer.
These vaccines are safe, as they did not induce unwanted side effects.

### DESCRIPTION OF THE INVENTION

Definitions

Within this specification, the expression “deletion of a gene” may be total or partial length deletion, providing that the exact alteration is not indicated.

According to the present invention, a “bacterial artificial chromosome” (BAC) is a DNA sequence comprising the F-factor sequence. Plasmids that contain this sequence, called F plasmids, can stably maintain heterologous sequences longer than 300 kb with a maximum of one or two copies per cell. The corresponding BACs can be any known in the state of the art.

The term “coronavirus” is used according to the present invention to refer to a group (“Family”) of viruses that has a simple linear single-stranded RNA ssRNA molecule, positive sense, from 25 to 33 kb. These viruses typically contain 4 to 10 structural genes.

The term coronavirus includes any member of the Coronaviridae family, preferably Orthocoronaviridae, and more preferably of the genus Betacoronavirus and even more preferably MERS-CoV.

As used herein “genes encoding genus-specific accessory proteins” are those genes of the coronavirus genome that encode the synthesis of proteins that are not incorporated into the structure of the virus.

In the present invention, the expression “virulence genes” are all those genes included in the genome of the coronavirus whose deletion attenuates the virus, and that, generally, do not have a structural function.

Herein “expression vector” can be a bacterial artificial chromosome (BAC), a cosmid and/or a derived artificial chromosome P1.

The term “nucleic acid” as used in this description includes genes or gene fragments, as well as, in general, any DNA or RNA molecule.

As used herein, the term “replicon” is synonymous with “replicon RNA” and “replicon RNA” and refers to an RNA that is self-amplifying (since it can make many copies of itself), but defective in propagation. This replicon can even form virus-like particles (VLPs), formed from subgenomic RNAs that act as messenger RNAs and are translated into proteins that are assembled into structures giving rise to VLPs, which contain the replicon RNA inside them.

As used herein, the term “VLP-E+” (functional, in the sense that it gives rise to a replicon that is infective because of carrying the E protein) refers to the VLP generated by a replicon RNA, to which at least one of the deleted proteins has been added in trans.

In the present specification, the expression “inducing protection” should be understood as inducing an immune response in the recipient organism, mediated by an antigen encoded by the RNA replicon of the invention, generating a long-term memory effect.

This immune response may be increased by mechanisms that involve the induction of substances which enhance the humoral response mediated by antibodies, or cellular, mediated by interleukins, cytokines, interferons, or the like, and substances that mediate intracellular processes that provide immune protection against infectious agents.

In the present application the expression MERS-MA30-Δ[3-E] and MERS-MA30-Δ[3, 4a, 4b, 5, E] are synonymous and are used interchangeably.

In the present application the expression MERS-CoV-Δ[3-E] and MERS-CoV-Δ[3,4a, 4b, 5,E] are synonymous and are used interchangeably.

The terms “vaccine” and “vaccine composition” are synonyms and have the usual meaning in the field.

Herein the number “4” within the name of an RNA replicon or an attenuated virus comprises genes 4a and 4b.

The present invention relates to an RNA replicon of a coronavirus in which it has been deleted:
partially the gene encoding protein E and
total or partially at least 4 genes encoding genus-specific accessory proteins selected among proteins 3, 4a, 4b and 5 of MERS-CoV.

The RNA replicon from MERS-CoV can have an identity of at least 55%, preferably 65%, more preferably 75%, even more preferably 85%, at least 90% identity, and even more preferably 91% or 92%. or 93% or 94% or 95% or 96% or 97% or 98% or even up to 99% with respect to the replicon sequence SEQ_ID 1, specifically with respect to the fragment of the attached sequence comprised from nucleotides 7890 to 35838.

In a preferred embodiment, the polynucleotide sequence of the MERS-CoV-Δ[3-E] replicon of the invention is described in SEQ_ID 1, specifically from nucleotides 7890 to 35838, of the attached sequence list.

To study the use of RNA replicons derived from coronaviruses as vaccines, the use of animal models is necessary. The most widely used animal model in clinical research, the mouse (Mus musculus), is not susceptible to infection by MERS-CoV, since the S protein of MERS-CoV does not recognize the homologous murine protein of the human receptor. A derivative of MERS-CoV that is pathogenic in these humanized animals has been used. From the human pathogenic MERS-CoV virus sequence Genbank JX869059, an infective strain was generated that caused the death of all infected mice, by passage of said MERS-CoV virus for 30 consecutive times in mice (Li et al., 2017). This sequence was deposited in GeneBank under accession number MT576585.

In a particular embodiment, the gene that encodes protein E and, in addition, the genes that encode three genus accessory proteins 4a, 4b and 5 proteins were totally or partially deleted, from the genome of a mouse-adapted MERS-CoV (MERS-MA30) (Li et al, 2017), to obtain an RNA replicon. This RNA replicon from MERS-MA30-Δ[3-E] may have at least 55% identity, preferably 65%, more preferably 75%, even more preferably 85%, at least 90% identity, and still more preferably 91% or 92% or 93% or 94% or 95% or 96% or 97% or 98% or even up to 99% with respect to the replicon sequence SEQ_ID 2, and specifically with respect to the fragment of the sequence comprised from nucleotides 7890 to 35838.

In a preferred embodiment, the polynucleotide sequence of the MERS-MA30-Δ[3-E] replicon of the invention is described in SEQ_ID 2, specifically from nucleotides 7890 to 35838, of the attached sequence list.

SEQ_ID 1 has a percentage of identity with SEQ_ID 2, specifically from nucleotides 7890 to 35838 of both sequences, of 99.96%. Therefore, it can be concluded that the results in mouse models obtained with the mouse-adapted MERS-CoV replicon, can be extrapolated to those that would be obtainable with the MERS-CoV replicon in humans.

The MERS-CoV-derived RNA replicon comprises at least the genes encoding the 1a, 1ab, S, M, and N proteins.

In another particular embodiment, at least one in-frame deletion of the gene encoding the nspl protein has been made in the RNA replicon. Preferably, nucleotides 792 to 827 of the ORF1a gene (nsp1-ΔD) are deleted, and/or nucleotides 708 to 734 (nsp1-ΔC) can be deleted, and/or at least a small deletion can be made between 27 and 36 nt, between positions 528 and 848 of the coronavirus genome.

The nsp1 protein is a modulator of the antiviral response and small deletions inside this gene have been shown to completely attenuate coronaviruses (Jimenez-Guardeño et al, 2015).

These modifications will act as a safety system in the event that the replicon could evolve restoring the virulence of the original wild-type MERS-CoV.

The RNA replicon can be modified by substituting at least one nucleotide with a modified nucleotide selected from pseudouridine and methylpseudouridine or similar alternatives in order to increase stability, translatability, and reduce activation of the innate immune response.

The nucleotide sequence of the RNA replicon nucleic acid may be optimized, in at least one of the codons of the genes it comprises. Codon optimization involves the introduction of silent point mutations into the codons of the polynucleotide sequence, to facilitate its expression and translation into proteins in a specific host. Preferably the host is a mammal, or more preferably the host is a human. One person skilled in the art would know that the number of optimized codons depends on both the polynucleotide sequence and the organism in which it is going to be expressed.

In a particular embodiment, the nucleotide sequence of the S protein gene has been optimized, specifically 50% of the codons of said gene have been optimized.

Likewise, the sequence of nucleotides or codons of any gene, or of a specific region of a gene, may be optimized for expression in cells of a host in at least 10% of the codons, preferably 20%, more preferably a 30%, even more preferably 40%, even more preferably 50% of the codons, even more preferably 60%, even more preferably 70%, even more preferably 80%, even more preferably 90%. The sequence of polynucleotides is preferably at least one replicon gene, e.g., the S, M, N and ORF1 ab gene.

Codon optimization can be performed following a standard protocol in the field. To determine which are the optimized codons, any computer tool can be used, such as Design Vector. Then the polynucleotide fragments are produced with the optimized codons by chemical synthesis. A person skilled in the art would know how to optimize the nucleotide or codon sequence of any gene, or of a particular region of a gene.

In a particular embodiment of the replicon, the sequence of the gene that encodes the S protein of the MERS-MA30-CoV coronavirus is SEQ_ID 3, whereas the sequence of the gene that encodes the S protein without the optimized codons is SEQ_ID 4.

In another particular embodiment of the replicon, the sequence of the gene that encodes the S protein of the MERS-CoV coronavirus is SEQ_ID 5, whereas the sequence of the gene that encodes the S protein without the optimized codons is SEQ_ID 6.

MERS-CoV protein S is a trimeric protein that exists in a metastable, prefusion conformation, undergoing structural rearrangement to fuse with the host cell plasma membrane. It has been shown that the prefusion conformation has higher antigenicity.

The prefusion state is stabilized by point mutations: V1060P and L1061P in the amino acid sequence of protein S (Pallesen et al, 2017). These positions are common regardless of whether the virus is MERS-CoV or MERS-MA30.

The RNA replicon of the invention can have a size between 18 and 29 kb, preferably between 20 and 27 kb, more preferably between 22 and 26 kb and even more preferably between 22 and 24 kb.

In another particular embodiment, the sequence of the gene encoding protein S has at least one of the following modifications: V1060P and L1061 P, in the polynucleotide sequence these modifications are 24633_24634 delins CC and 24637_24638 delins CC. Respectively, in both cases two consecutive nucleotides have been modified.

In another particular embodiment, the sequence of the gene that encodes the S protein of MERS-MA30-CoV has the codons optimized for its expression in mammalian cells and the modifications 24633_24634 delins CC and 24637_24638 delins CC being the resulting sequence of the S gene SEQ_ID 7.

In another particular embodiment, the sequence of the gene that encodes the S protein of MERS-CoV has the codons optimized for its expression in mammalian cells and the modifications 24633_24634 delins CC and 24637_24638 delins CC, being SEQ_ID 8 the resulting sequence of the S gene.

In a particular embodiment, in the RNA replicon of the invention has been deleted:
partially the gene encoding protein E and
total or partially at least four genes that encode genus accessory proteins selected among genes 3, 4a, 4b and 5 of MERS-CoV,

in addition,
the nucleotide sequence of the S protein of the indicated replicon has the codons optimized for its expression in mammals,
the sequence of the gene that encodes S protein has, at least, the following modifications: V1060P and L1061 P, and
has a deletion of 35 nucleotides in the gene that encodes protein nsp1 (nsp1-ΔD).

This replicon is named V1-CD, and the polynucleotide sequence of this mouse-adapted replicon is described in SEQ_ID 9 (MERS-MA30-V1-CD). The polynucleotide sequence of this human-adapted replicon is described in SEQ_ID 10 (MERS-CoV-V1-CD)

SEQ_ID 9 has an identity percentage with SEQ_ID 2, specifically from nucleotides 7890 to 35838, of up to 96.12%. SEQ_ID 2 is the sequence of the mouse-adapted MERS-MA30-Δ[3-E] replicon.

SEQ_ID 10 has a % identity with SEQ_ID 1, specifically from nucleotides 7890 to 35838, of up to 96.03%. SEQ_ID 1 is the replicon sequence MERS-CoV-Δ [3,4a,4b,5,E].

In another particular embodiment, the RNA replicon of the invention has been deleted:
partially the gene encoding protein E and
total or partially at least 4 genes that encode genus-accessory proteins selected among genes 3, 4a, 4b and 5, in the case of MERS-CoV,

in addition,
the nucleotide sequence of the S protein of a given replicon has the codons optimized for expression in mammals, and
has a 35 nucleotide deletion in the gene encoding the nsp1 protein (nsp1-ΔD).

This replicon is designated V1-VLP and the polynucleotide sequence of this mouse-adapted replicon is SEQ_ID 11 (MERS-MA30-V1-VLP). The polynucleotide sequence of this human-adapted replicon is SEQ_ID 12 (MERS-CoV-V1-VLP).

SEQ_ID 11 has at least 96.11% identity with SEQ_ID 2, specifically from nucleotides 7890 to 35838. SEQ_ID 2 is the sequence of the mouse-adapted MERS-MA30-Δ[3-E] replicon.

SEQ_ID 12 has a % identity with SEQ_ID 1, specifically from nucleotides 7890 to 35838, of 96.04%. SEQ_ID 1 is the replicon sequence MERS-CoV-Δ[3,4a,4b,5,E]

The identity percentage between SEQ_ID 9 and SEQ_ID 11 is 99.8%.

The identity percentage between SEQ_ID 10 and SEQ_ID 12 is 99.98%.

It is to be understood that the nucleic acids of the invention may be single-stranded or double-stranded, and further contain a nucleotide sequence complementary to the nucleotide sequence of the nucleic acid of the invention. The term “complementary” refers to the ability of two single-stranded polynucleotide fragments to form base pairs with each other. Substantially complementary polynucleotide fragments may include at least one base pair mismatch such that at least one nucleotide present in a first polynucleotide fragment will not pair with at least one nucleotide present in a second polynucleotide fragment, however, the two polynucleotide fragments will still have the capacity to hybridize. Therefore, the present invention encompasses polynucleotide fragments that are substantially complementary. Two polynucleotide fragments are substantially complementary if they hybridize under hybridization conditions exemplified by 2×SSC (SSC: 150 mM NaCl, 15 mM trisodium citrate, pH 7.6) at 55° C. Substantially complementary polynucleotide fragments for purposes of the present invention preferably share at least about 85% nucleotide identity, preferably at least about 90% or 95% or 99% nucleotide identity.

The locations and levels of nucleotide sequence identity between two nucleotide sequences can be determined by means of the “Clustal” software available from the European Bioinformatics Institute (EBI) or “BLAST” available from the National Center for Biotechnology Information (NCBI).

For its administration, the RNA replicon can be wrapped by one of the following options:
combination with a polymer selected to cover it, for example, among: chitosan, polyplex, polyethyleneimine (PEI), poly(lactic acid-co-glycol) (PLGA), cyclodextrin (CD), dendrimers (poly-amidoamine) PAMAM, poly-propylenimine (PPI) and derivatives thereof,
the combination with at least one lipid nanoparticle, for example from: permanently charged cationic lipids, ionizable cationic lipids and combinations thereof: 1,2-di-O-octadecenyl-3-trimethylammoniopropane (DOTMA), 1,2-dioleoyl-sn-glycero-3-phosphoethanolamine (DOPE), 1,2-dilinoleyl-3-dimethylammoniopropane (DLinDAP), 1,2-dilinoleyloxy-3-dimethylaminopropane (DLinDMA), 2,2-dilinoleyl-4-dimethylaminoethyl-[1,3]-dioxolane (DLin-KC2-DMA), and O-(Z,Z,Z,Z-heptatriaconta-6,9,26,29-tetraen-19-yl)-4-(N,N-dimethylamino) (DLin-MC3-DMA) and 1,2-dioleoyl-3-dimethylammoniopropane (DODAP), or other lipids similar to the ones mentioned.
Other systems based on peptides or carbohydrates, for example: peptides in the α-Helix or β-Sheet conformation, linear peptides such as homodipeptide (phe-phe) or Aβ-amyloid, cyclic peptides, such as cyclo-(L-Gln-D-Ala-L-Glu-D-Ala)₂, amphiphilic peptides, or peptides with specific amino acid sequences that facilitate cell penetration, such as the TAT dodecapeptide GRKKRRQRRRPQ, or peptides capable of interact with overexpressed membrane receptors, for example for the DPP4 receptor. In the case of carbohydrates, derivatives of glucose, mannose and galactose such as N-(D-glucos-1-yl)-L-asparagine, N-(D-fructos-2-yl)-L-asparagine, N-(D-glucos-1-yl)-L-glutamine, N-(D-glucos-1-yl)-L-methionine, can be used.

In a particular embodiment, the RNA replicon of the invention may be wrapped for administration in a VLP-E+, which includes the E protein provided in trans and confers replicon transmissibility to the virus, as the RNA replicon does not include the gene that encodes protein E; this type of VLPs differs from VLP-E−, that is, those replicons that do not include the E protein. This particular embodiment is not possible for the RNA replicon that has point mutations in the S protein gene to stabilize the prefusion state.

The RNA replicons provided by the invention are replication competent, but propagation deficient, what prevents them from being transmitted from cell to cell. Upon infecting a cell, the RNA replicon of the invention is amplified and consequently increases its ability to express various virus proteins. However, these proteins form a VLP-E that can wrap around (encapsidate) the RNA replicon, do not disseminate to other cells in the body; said VLPs-E are recognized by the host's immune system. Among the VLP proteins produced by the replicon of the invention is the spike protein (S) of coronaviruses, which is the largest viral antigen that induces protective neutralizing antibodies, as well as virus-specific T cells.

Another aspect of this invention relates to a functional VLP-E+ comprising a replicon RNA as defined above. This functional VLP-E+ contains at least one of the proteins encoded by one of the genes deleted in the RNA replicon, preferably the E protein.

The RNA replicon inside a VLP-E+ cannot encode the S protein in the prefusion state, stabilized by the point mutations: V1060P and L1061P, as this fixed structure would be unable to facilitate the entrance of the virus in cells, since the mechanism of the VLP-E+ of the invention to enter cell mimics that of a natural infection. VLP entry into the cell is mediated by protein S, and requires conformational changes that lead virus and cell membrane fusion, an even incompatible with the prefusion state of protein S.

Both the V1-CD replicon or the V1-VLP replicon, can be administered by a chemically defined formulation. Preferably, the V1-CD replicon will be used. In this replicon, the S protein is blocked in a conformation that prevents the natural entry of the virus into the cells increased it safety.

In contrast, the V1 CD replicon described above is not suitable for delivery via a VLP-E+.

Another aspect of this invention relates to a chemically defined (CD) formulation comprising an RNA replicon as defined above and at least one of the following type of compounds:
A polymer selected from chitosan, polyplex, polyethyleneimine (PEI), poly(lactic-co-glycolic acid) (PLGA), cyclodextrin (CD), dendrimers (polyamidoamine) PAMAM, polypropylenimine (PPI), and derivatives thereof.
a lipid nanoparticle, selected for example from: permanently charged cationic lipids, ionizable cationic lipids and combinations thereof: 1,2-di-O-octadecenyl-3-trimethylammoniopropane (DOTMA), 1,2-dioleoyl-sn-glycero-3-phosphoethanolamine (DOPE), 1,2-dilinoleyl-3-dimethylammoniopropane (DLinDAP), 1,2-dilinoleyloxy-3-dimethylaminopropane (DLinDMA), 2,2-dilinoleyl-4-dimethylaminoethyl-[1,3]-dioxolane (DLin-KC2-DMA) and O-(Z,Z,Z,Z-heptatriaconta-6,9,26,29-tetraen-19-yl)-4-(N,N-dimethylamino) (DLin-MC3-DMA) and 1,2-dioleoyl-3-dimethylammoniopropane (DODAP), or alternative lipids similar to these
Other systems based on peptides or carbohydrates, for example: peptides in the α-Helix or β-Sheet, linear peptides such as homodipeptide (phe-phe) or Aβ-amyloid, cyclic peptides, such as cyclo-(L-Gln -D-Ala-L-Glu-D-Ala)₂, amphiphilic peptides, or peptides with specific amino acid sequences that facilitate cell penetration, such as the TAT dodecapeptide GRKKRRGRRRPG, or peptides capable of interact with overexpressed membrane receptors, for example for the DPP4 receptor. In the case of carbohydrates, derivatives of glucose, mannose and galactose can be used, such as, for example, N-(D-glucos-1-yl)-L-asparagine, N-(D-fructos-2-yl)-L-asparagine, N-(D-glucos-1-yl)-L-glutamine, N-(D-glucos-1-yl)-L-methionine.

The RNA replicon within a chemically defined formulation can encode an S protein, which includes in its sequence the point mutations: V1060P and L1061 P, which fix the molecular structure of this protein in a conformation that blocks the natural entry of the virus into the cells. cells, what increases the safety of the replicon RNA. This is possible in the variant of the vaccine based on the chemically defined replicon, because its entry is not dependent on protein S, but is mediated by one of the systems mentioned above (for example, a polymer) that protects the RNA and facilitates its entry into the cell.

The invention additionally provides a method for the design and construction by reverse genetics (genetic engineering) of RNA replicons derived from the genomes of coronaviruses for the construction of vaccines that provide protection against infection by the coronaviruses from which they are derived, and also from other related coronaviruses (Almazán et al., 2013; Almazán et al., 2015; Sambrook and Russell, 2001).

Another aspect of the present invention relates to a method for preparing a coronavirus replicon RNA comprising:
i. the construction of the full-length cDNA from the gRNA of a coronavirus and its insertion into an expression vector, obtaining an infectious clone
ii. the deletion of:
   part of the gene encoding protein E and
   full or partial deletion of at least 4 genes encoding accessory proteins selected among proteins 3, 4a, 4b and 5, in the case of MERS-CoV.
iii. transfecting the expression vector from the previous step into a host cell, under suitable conditions for its expression.

The nucleotide sequence of the full-length cDNA may exhibit at least 50% identity, preferably 60%, more preferably 70%, even more preferably 80%, even more preferably 90%, and even more preferably a 91% or 92% or 93% or 94% or 95% or 96% or 97% or 98% or 99% identity to the gRNA polynucleotide sequence of a coronavirus.

The nucleotide sequence of the full-length cDNA may have an identity of at least 50%, preferably 60%, more preferably 70%, even more preferably 80%, even more preferably 90%, and even more preferably 91%, or 92% or 93% or 94% or 95% or 96% or 97%, or 98% or 99% identity with respect to the polynucleotide sequence corresponding to MERS-CoV (Genbank: JX869059).

The method of the invention may comprise the partial deletion of the gene that encodes protein E and the total or partial deletion of the genes that encode genus accessory proteins 3, 4a, 4b and 5, in the case of MERS-CoV.

The method may comprise the modification of the cDNA sequence by means of substitutions, deletions, additions or any other modification in the nucleic acid sequence, prior to the total or partial deletion of genes to obtain the RNA replicon of the invention.

The method may also comprise the replacement of at least one nucleotide, for another one that is chemically or enzymatically modified.

A full-length cDNA can be obtained by any method known in the art. Due to the length of the cDNA it is possible to obtain several fragments by, for example, chemical synthesis and to introduce each of these fragments into a vector. The polynucleotide sequence of these fragments can be modified preferably in their ends sequence, in order to introduce restriction sites that facilitate their subsequent combination to obtain the full-length infective clone in a single expression vector. These expression vectors can be any wherein the full-length cDNA will fit into, such as a bacterial artificial chromosome.

Prior to the total or partial deletion of the genes to obtain the RNA replicon of the invention, the expression vector comprising the full-length cDNA of the coronavirus gRNA can be transfected into appropriate cells. These cells will produce recombinant virions of that coronavirus. These recombinant virions have the same ability to replicate and spread as the whole virus. The cell to use could be hamster kidney (BHK21), African green monkey (Vero-81) cells, human cells derived from kidney (HEK293), liver (Huh-7), or lung (Calu3, Calu3 2B4, MRC-5). The growing conditions, as well as the recovery of infectious virions can be performed by any method known in the state of the art (Almazán et al., 2013).

The strategies to totally or partially delete the genes of the coronavirus genome can be any of the state of the art, for example, use of restriction enzymes, recombination between vectors and CRISPR technology (Almazán et al., 2015; Sambrook and Russell, 2001).

The expression vector with the full-length cDNA may include all the regulatory elements that allow the expression of the full-length RNA in a suitable cell to obtain a recombinant coronavirus.

The expression vector of the method must have the appropriate elements for its replication and expression. The use of the cytomegalovirus (CMV) immediate early (IE) promoter for expression in mammalian cells or the T7 promoter is preferred.

Other promoters that can be used for its expression in mammalian cells are, for example, the human Ubiquitin C (UBC) promoter and the PGK promoter. The advantage of these promoters is that subsequent approval for therapeutic use is facilitated, since these sequences are naturally occurring in humans. For example, the promoters described in the scientific articles: How to Choose the Right Inducible Gene Expression System for Mammalian Studies? Kallunki T, et al Cells. 2019 Jul. 30; 8(8):796 and/or Mammalian cell protein expression for biopharmaceutical production. Zhu J. Biotechnol Adv. 2012 September-October; 30(5):1158-70, can be used

The expression vector of the method can also have the elements suitable for its expression in vitro, that is, in the absence of cells. In this case, the replication and expression plasmids may comprise the sequences necessary for in vitro amplification and transcription under the control of the T7 promoter.

The expression vectors containing the replicons of the invention may carry, for example, the CMV or T7 promoter. If the first one is used, transcription is carried out inside the cell, by cellular poly II polymerase. The resulting viral RNA will replicate in the cell, will express viral proteins and form VLPs. If T7 promoter is used, the cDNA encoding this promoter including the replicon sequences will be transcribed in vitro. In order to introduce the cDNA into cells, a vehicle such as those mentioned previously is required. The T7 promoter is appropriate for the V1-CD replicon.

The 5′ end of the expression vector may contain the sequences indicated below, or variants thereof:

T7P-5′UTR-MISC-DLP-P2A-3′ UTR wherein UTR stands for untranslated region,

MISC is a sequence that comes from the replicon of the Venezuelan equine encephalitis virus (VEEV),

DLP is a sequence that comes from the Sindbis virus (part of the Sindbis virus RdRp),

P2A is Porcine Teschovirus-1 protease 2a, the corresponding polynucleotide sequences of these elements being as follows:

T7P (SEQ_ID 13):
TAATACGACTCACTATAG
5′UTR (SEQ_ID 14):
ATAGGCGGCGCATGAGAGAAGCCCAGACCAATTACCTACCCAAA
MISC (SEQ_ID 15):
TAGGAGAAAGTTCACGTTGACATCGAGGAAGACAGCCCATTCCTCAGAGC
TTTGCAGCGGAGCTTCCCGCAGTTTGAGGTAGAAGCCAAGCAGGTCACTG
ATAATGACCATGCTAATGCCAGAGCGTTTTCGCATCTGGCTTCAAAACTG
ATCGAAACGGAGGTGGACCCATCCGACACGATCCTTGACATTGGA
DLP (SEQ_ID 16):
ATAGTCAGCATAGTACATTTCATCTGACTAATACTACAACACCACCACCA
TGAATAGAGGATTCTTTAACATGCTCGGCCGCCGCCCCTTCCCGGCCCCC
ACTGCCATGTGGAGGCCGCGGAGAAGGAGGCAGGCGGCCCCG
P2A (SEQ_ID 17):
GGAAGCGGAGCTACTAACTTCAGCCTGCTGAAGCAGGCTGGAGACGTGGA
GGAGAACCCTGGACCT

The 3′ end of the expression vector may contain the sequences or variants thereof:

3′ UTR- Poly A - terminador T7
3′UTR (SEQ_ID 18):
ATACAGCAGCAATTGGCAAGCTGCTTACATAGAACTCGCGGCGATTGGCA
TGCCGCTTTAAAATTTTTATTTTATTTTTCTTTTCTTTTCCGAATCGGAT
TTTGTTTTTAATATTTC
PolyA (SEQ_ID 19):
AAAAAAAAAAAAAAAAAAAAAAAAAAAAAAAAAAAAAAAA
terminator T7(SEQ_ID 20):
CCCCTCTCTAAACGGAGGGGTTTTTTT.

Another aspect of the present invention relates to an expression vector whrein the cDNA sequence complementary to the RNA replicon derived from a coronavirus, preferably MERS-CoV, has been inserted.

In a particular embodiment, the cDNA sequence complementary to the RNA replicon inserted in the expression vector has an identity of at least 55%, preferably 65%, more preferably 75%, even more preferably 85%, even more preferably 90%, and even more preferably 91% or 92% or 93% or 94% or 95% or 96% or 97% or 98% or 99% identity with respect to the sequence SEQ_ID 1, specifically with respect to the fragment of the indicated sequence comprised from nucleotides 7890 to 35838.

In another particular embodiment, the cDNA sequence complementary to the RNA replicon inserted in the expression vector has an identity of at least 55%, preferably 65%, more preferably 75%, even more preferably 85%, even more preferably 90%, and even more preferably 91% or 92% or 93% or 94% or 95% or 96% or 97% or 98% or 99% identity with respect to the sequence SEQ_ID 2, specifically with respect to the fragment of the indicated sequence comprised from nucleotides 7890 to 35838.

In another particular embodiment, the cDNA sequence complementary to the RNA replicon inserted in the expression vector has an identity of at least 55%, preferably 65%, more preferably 75%, even more preferably 85%, even more preferably 90%, and even more preferably 91% or 92% or 93% or 94% or 95% or 96% or 97% or 98% or 99% identity with respect to the sequence SEQ_ID 9, SEQ_ID 10, SEQ_ID 11, or SEQ_ID 12.

This expression vector can be selected from a bacterial artificial chromosome (BAC), a cosmid and a derived artificial chromosome P1.

The expression vector of the invention encodes proteins that can be expressed in cell cultures or in mammalian cells, including an experimental animal model, such as humanized transgenic mice for the DPP4 receptor of the virus. The organism or cell may be eukaryotic or prokaryotic, and may be a bacterium, a yeast, a protozoan, or animals such as an insect, a human, a bird, or a non-human mammal, such as a cat.

The expression vector comprising the cDNA sequence complementary to the RNA replicon is a DNA molecule that has an origin of replication and is therefore capable of replicating in a suitable cell. The vector used is suitable for maintaining and amplifying the RNA replicon of the invention in a suitable host cell, such as a bacterium, for example Escherichia coli. The expression vector generally comprises a selection system for cells carrying said vector, for example:
an antibiotic resistance gene that allows the selection of cells that carry it: for example, genes for resistance to chloramphenicol (chloramphenicol acetyl transferase, cat), kanamycin or neomycin,
a selection system based on the complementation of auxotrophic markers, provided that a bacterial strain deficient for a metabolic pathway is used, for example, an alteration in the DAP (diaminopimelic acid) pathway due to a mutation or deletion in the DapD gene or the use of a ΔTriA strain, which has a low growth rate with glucose as carbon source and no growth with glycerol. Only the strain carrying the plasmid expressing the tpiA gene can restore normal growth.
a toxin/antitoxin mechanism, for example, the hok/sok or the ccdB/ccdA system,
a ColE1 based repression mechanism (Mairhofer and Grabherr, 2008; Mairhofer et al., 2008),
a mechanism based on the sacB counterselection marker (WO2010/135742).

The introduction of the expression vector containing the complementary cDNA sequence to the RNA replicon of the invention in the host cell can be carried out by any means known in the state of the art to transfect plasmids, preferably by lipofectation, calcium phosphate, or electroporation.

The cDNA of the replicon of the invention is inserted between the 5′ and 3′ elements of the expression vector.

The minimum requirement necessary for transcription of the replicon cDNA of the invention is the T7 promoter (T7P), when transcription is performed in vitro in the chemically defined manner, or the cytomegalovirus promoter when the replicon is expressed inside cells.

In a particular embodiment, the cDNA sequence complementary to the RNA replicon of the invention that has been inserted into the expression vector is flanked at the 3′ end by the following elements and in this order: a poly (A) tail of, at least 24 adenine residues, the ribozyme sequence of the hepatitis delta virus (HDV), and bovine growth hormone (BGH) termination and polyadenylation sequences (Almazán 2013).

The plasmid must be capable of linearizing (cutting the circular plasmid DNA encoding the RNA replicon by means of, for example, restriction enzymes) prior to RNA synthesis, so a unique restriction enzyme cutting site has to be introduced, after the termination sequence of phage T7.

Termination sequences for the phage T7 polymerase along the replicon sequence of the invention, such as the ATCTGTT sequence, should be avoided, hence, sequences of this type along the replicon should be mutated without affecting the replicon functionality.

The RNA replicon resulting from the deletion of at least five genes of the coronavirus from which it comes, preferably MERS-CoV, can be expressed in a suitable cell line, for example, a cell line that provides one of the deleted proteins in trans, that allows the RNA replicon to wrap itself in a functional VLP, preferably the V1-VLP replicon. Cells suitable for expressing the RNA replicon wrapped in a functional VLP-E+ are, for example, BHK21, Huh-7, HEK293, Calu3, Calu3 2B4, MRC-5 and Vero-81. The RNA replicon can be combined with a polymer or a lipid nanoparticle, or other nanoparticle, preferably the V1-CD replicon.

The cell lines suitable for the expression of the invention must be previously modified to be able to provide in trans at least one of the genes deleted in the replicon of the invention, preferably the gene that encodes protein E.

In an additional embodiment, starting from the expression vector in which the cDNA sequence complementary to the RNA replicon of the invention has been inserted, an

RNA replicon can be generated divided into at least two fragments (that is, two components that encode different viral proteins and therefore they are two expression vectors). Each of these vectors includes a partial fragment of the cDNA sequence complementary to the RNA replicon of the invention. The fragments of the cDNA sequence complementary to the RNA replicon of the invention, which have been inserted into at least two expression vectors may be overlapping. One of the fragments, the one that includes the replicase gene, is autonomous for its replication, while the other(s) fragment(s), depend on the first one (autonomous for its replication), because this is the only one that includes the replicase gene.

The set of at least 2 expression vectors, as explained in the previous paragraph, comprises: The replicase gene and the N protein gene included in the sequence SEQ_ID 2 or SEQ_ID 1, SEQ_ID 9, SEQ_ID 10, SEQ_D 11, SEQ_ID 12, and a second expression vector that includes sequences of the rest of the structural genes and/or sequences of non-essential genes included in SEQ_ID 2 or SEQ_ID 1: the fragment of the sequence from nucleotides 7890 to 35668 or SEQ_ID 9, SEQ_ID 10, SEQ_ID 11, or SEQ_ID 12, respectively.

In each of the expression vectors that comprise a cDNA fragment complementary to the RNA replicon of the invention, said fragment is flanked at its ends by the untranslated sequences (Untranslated Regions, or UTRs) of the 5′ end (5′ -UTR) and 3′ end (3′-UTR) of the coronavirus from which the nucleic acid of the replicon of the invention has been obtained, preferably MERS-CoV.

The genes of each fragment are preceded by the transcription-regulating sequences of each of them (transcription-regulating sequences, or TRSs) that control the expression of the corresponding messenger RNAs.

The strategies to obtain expression vectors with a partial fragment of the cDNA sequence complementary to the RNA replicon of the invention, can be carried out by any method known in the state of the art, preferably by a combination of nucleic acid fragments obtained by chemical synthesis and use of restriction enzymes.

To obtain the resulting replicon RNA, expression vectors containing partial fragments of cDNA sequences encoding all the genes that have not been deleted, can be expressed in a suitable cell line. The appropriate cell line is one that provides in trans one of the deleted proteins that allows the RNA replicon to wrap itself in a functional VLP-E+ for later use.

Suitable cell lines may be BHK21, Huh-7, HEK293, Calu3, Calu3 2B4, MRC-5 and Vero-81.

The RNA replicon can be combined with a polymer or a lipid nanoparticle.

To obtain the V1-CD replicon, the RNA coming from the pBAC containing the replicon is transcribed in vitro under RNase-free conditions, this pBAC can be any vector known in the field, for example, a pBeloBAC plasmid modified to include the T7 promoter, required for in vitro transcription. To do this, the protocol previously described by Eriksson K. K. et al, 208, Methods in Mol. Biol. 454:237-254, with some modifications is used. Briefly, highly purified pBAC, linearized or not, is used as template. In vitro transcription is performed with commercial kits that contain the T7 polymerase, such as the RiboMAX Large Scale RNA production system from Promega, adding a cap analog (Ribo m7G cap analog, Promega). The reaction proceeds for 2 hours at 30° C. Subsequently, the template DNA is eliminated by digestion with RNase-free DNase.

The in vitro transcribed RNA is purified by LiCl precipitation, quantified using a nanodrop, and analyzed for quality on an agarose gel.

In a particular embodiment, an expression vector comprises the cDNA fragment complementary to the RNA replicon of the invention that encodes ORF 1a, ORF 1ab and the N gene, and a second expression vector comprises the cDNA fragment complementary to the RNA replicon of the invention encoding genes S, M and genes encoding genus accessory proteins that had not been deleted from the RNA replicon of the invention.

In another particular embodiment, an expression vector comprises the cDNA fragment complementary to the RNA replicon of the invention that encodes ORF 1a, ORF 1ab and a second expression vector comprises the cDNA fragment complementary to the RNA replicon of the invention that encodes the N, S, M genes and genes encoding genus accessory proteins that would not have been deleted from the RNA replicon of the invention.

In another particular embodiment, an expression vector comprises the cDNA fragment complementary to the RNA replicon of the invention that encodes ORF 1a, ORF 1ab and the S gene, and a second expression vector comprises the cDNA fragment complementary to the RNA replicon of the invention encoding genes N, M and genes encoding genus accessory proteins that have not been deleted from the RNA replicon of the invention.

The replicon of the invention may include one or more heterologous nucleic acids of interest. Said heterologous nucleic acid is selected from a gene and/or a fragment of a gene encoding a gene product of interest.

Any heterologous gene of interest can be inserted into the nucleic acids according to the present invention. Particularly preferred is the insertion of genes encoding peptides or proteins that are recognized as an antigen from an infectious or foreign (non-self) agent by the mammalian immune system. The heterologous gene may therefore encode at least one antigen suitable for inducing an immune response against an infectious agent, and/or at least encode a molecule that interferes with the replication of an infectious agent, and/or an antibody that provides protection against the infectious agent. Alternatively or additionally, the heterologous gene may encode an immune modulator, a cytokine, an immune response enhancer and/or an anti-inflammatory protein.

The heterologous nucleic acid that can be inserted into the replicon of the invention can be a gene or gene fragment encoding a protein, a micro-RNA, a peptide, an epitope or any gene product of interest (such as enzymes, cytokines, interleukins, etc.).

The heterologous nucleic acid can be inserted into the infective clone of the invention by conventional genetic engineering techniques, in any appropriate region of the cDNA, e.g. after ORF 1ab or between two genes, following the initiator codon (AUG) and in reading frame with that gene; or, alternatively, in the areas corresponding to other ORFs. In the construction of the RNA replicon of the invention it is essential that the insertion of the heterologous nucleic acid does not interfere with any of the basic viral functions necessary for self-amplification and wrapping of the replicon in a VLP, when these are necessary.

Another additional aspect of this invention is a vaccine composition that induces protection in a subject against infection caused by the MERS-CoV coronavirus, such that said vaccine composition comprises an RNA replicon derived from MERS-CoV described above, together with, optionally:
at least one pharmaceutically acceptable excipient and/or
at least one chemical or biological adjuvant or immunostimulator.

As an excipient, a diluent can be used, such as physiological saline and other similar saline solutions, or also polymers of a different nature that have been developed for this purpose and are commercially available.

Preferred chemical adjuvants comprise AS03 or Matrix-M, aluminum hydroxide, Quil A, suspensions of alumina gels and the like, as oily, mineral oil based, glycerides and fatty acid derivatives, and mixtures thereof.

Biological adjuvants can amplify the immune response induced by the vaccine of the invention. The biological adjuvants are selected from substances that enhance cell response (PRC), substances that enhance subpopulations of T helper cells (JM and Th2) such as interleukin-1 (IL-1), IL-2, IL-4, IL-5, IL-6, IL-12, interferon gamma (IFN-gamma), tumor necrosis factor (TNF), and similar substances, which can potentiate the immune response in vaccinated subjects. These immune response regulators could be used in vaccine formulations with aqueous or oily adjuvants. Other types of adjuvants that modulate and immunostimulate the immune response can also be used, such as MDP (muramyl dipeptide), ISCOM (Immuno Stimulant Complex) or liposomes.

The vaccine composition of the invention can be administered to a subject topically, intranasally, orally, subcutaneously or intramuscularly, preferably intranasally.

The subject is preferably a mammal, more preferably a human or a domestic animal, for example a dog or cat, although alternative subjects may be treated in the course of vaccine or disease research. The dose of vaccine to be administered to a subject depends on the species and size of the subject, the nature of the condition being prevented, and can be readily determined by one skilled in the art.

As of May 1, 2021, 610 complete sequences of the MERS-CoV genome from both Camelus spp and Homo sapiens had been deposited in GenBank, the identity of these 610 sequences is in a range between 74 and 100%. Therefore, it is expected that the replicon and the vaccine composition of the present invention can protect not only humans but other animals against infection of this coronavirus.

Another additional object of this invention comprises the RNA replicon defined above for its use in a vaccine composition.

Said attenuated RNA replicon expressing one or more structural genes of a coronavirus can be used as part of a vaccine composition. Also provided is the use of an attenuated RNA replicon expressing one or more coronavirus structural genes in the manufacture of a vaccine. The vaccine composition is designed for use in protecting a subject against infection by a coronavirus, preferably MERS-CoV.

In a particular embodiment, the vaccine composition of the invention is administered to the subject simultaneously together with a chemical or biological adjuvant or immunostimulator.

In another particular embodiment, the vaccine composition of the invention is administered before or after the chemical or biological adjuvant or immunostimulator.

The vaccines of this invention can be presented in liquid or lyophilized form and can be prepared by suspending the components of the vaccine composition in the excipient. These systems can be in lyophilized form, the excipient can be the reconstituent itself.

Alternatively, the vaccine compositions disclosed in this invention can be combined with other conventional vaccines.

A single administration of the vaccine composition may be sufficient to provide adequate immunization, but in alternative embodiments, more than one dose of vaccine may be administered. For example, a first dose may be followed by a booster dose after one week, two weeks, three weeks, four weeks, one month, two months, three months, four months, five months, six months, or longer intervals.

The invention is illustrated by the following examples which describe in detail the objects of the invention. These examples should not be considered as limiting the scope of the invention but rather as illustrative of it.

### BRIEF DESCRIPTION OF THE FIGURES

FIG. 1. Diagram of the genome of coronavirus MERS-CoV. The genome of MERS-CoV (EMC/2012 strain, GenBank JX869059) is represented. The letters above the boxes represent the viral genes: L, leader sequence; S, spike protein gene; E, envelope protein gene; M, membrane protein gene; N, nucleocapsid protein gene. The numbers or letters above the boxes indicate the genus-specific genes. ORF, open reading frame; An, poly-A tail. In the case of MERS-CoV, those proteins encoded by genes 3, 4a, 4b, and 5 are considered genus-specific proteins.

FIG. 2. Construction of an infective clone of MERS-MA30 adapted to grow in mice. (A) The figure shows the genetic structure of MERS-CoV indicating by letters and numbers the names of the genes [ORF1a, ORF1b, S, 3, 4a, 4b, 5, E, M, and N], wherein ORF stands for open reading frame. The boxes at the bottom of the bar indicate the positions of the mutations introduced into the genome of the MERS-MA30 SEQ_ID 21 virus adapted to grow in mice after 30 passages, which were not present in the human MERS-CoV non-adapted to grow in mice. The MERS-CoV cDNA genome has been described in GenBank JX869059.

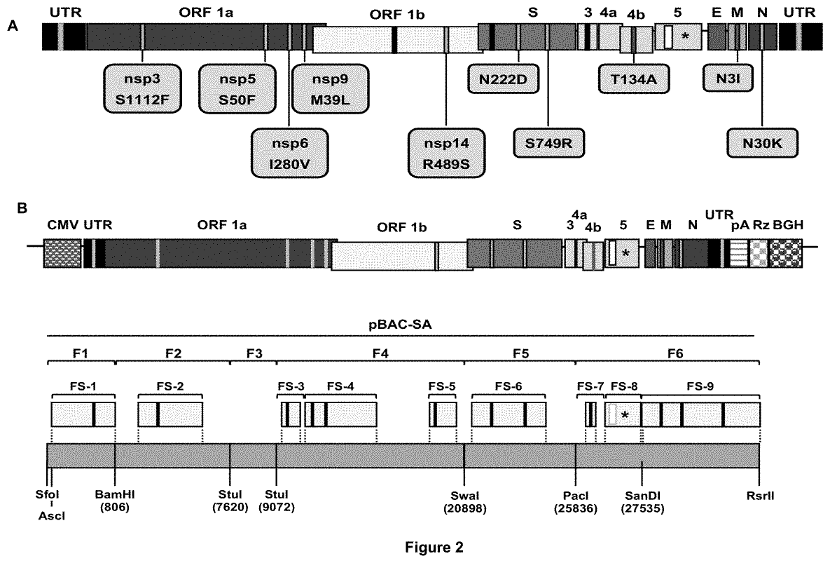

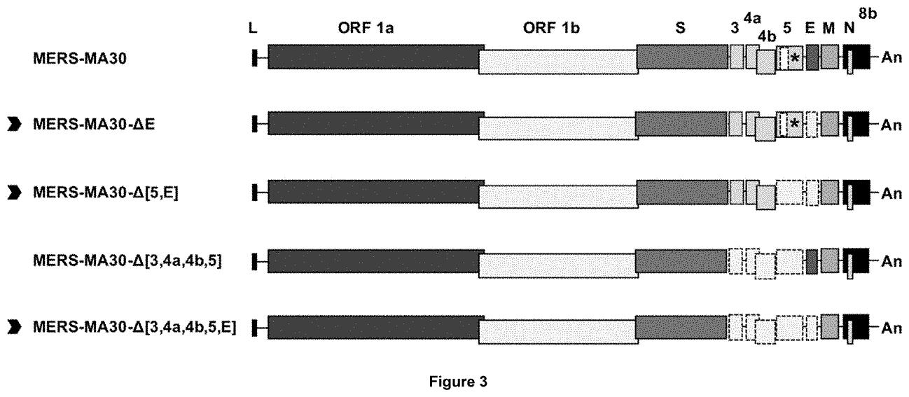

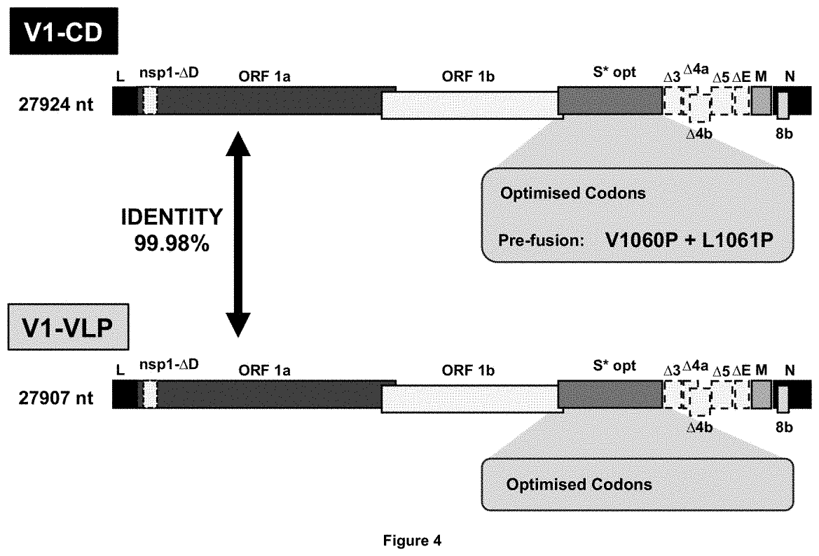

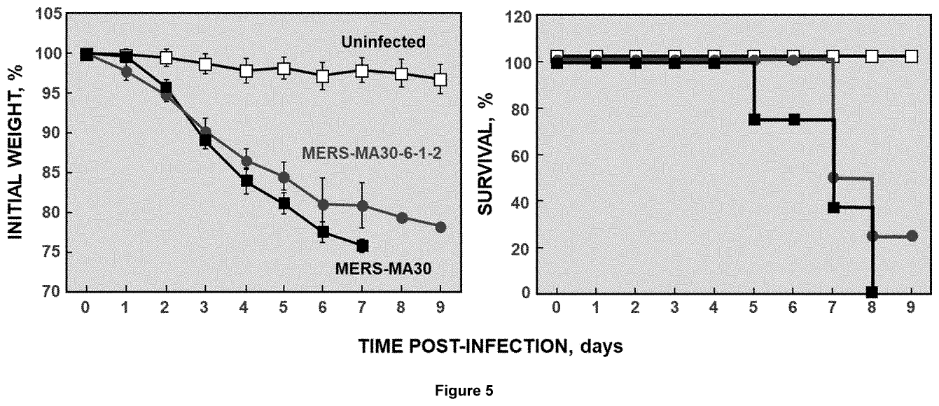

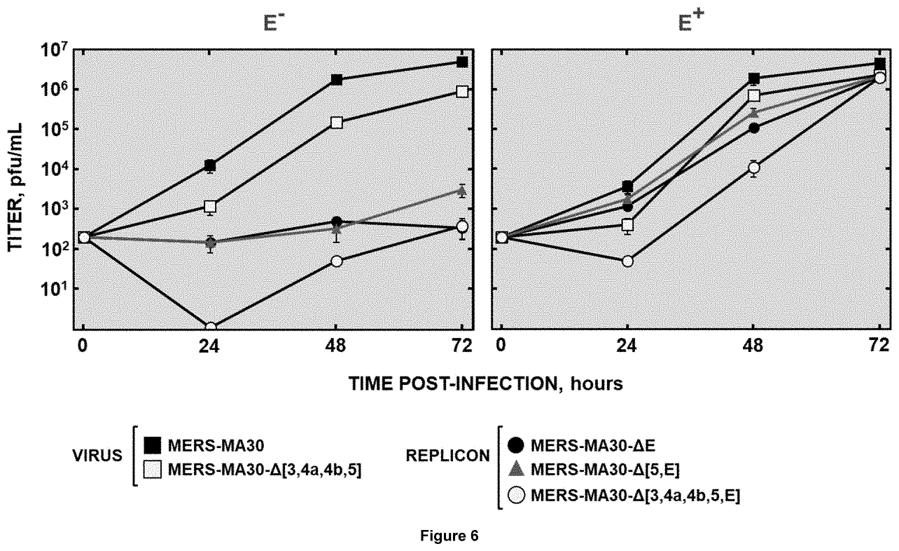

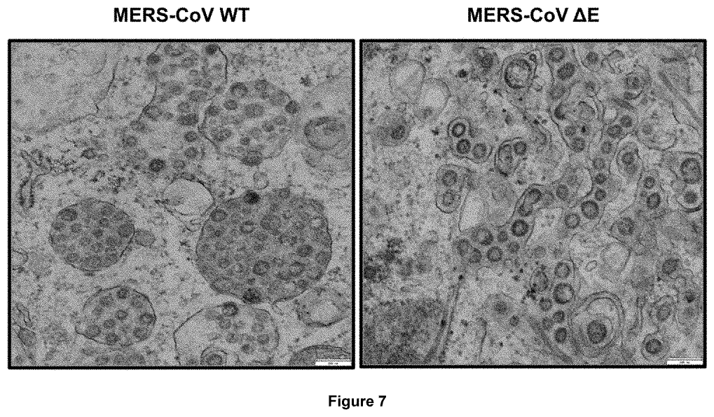

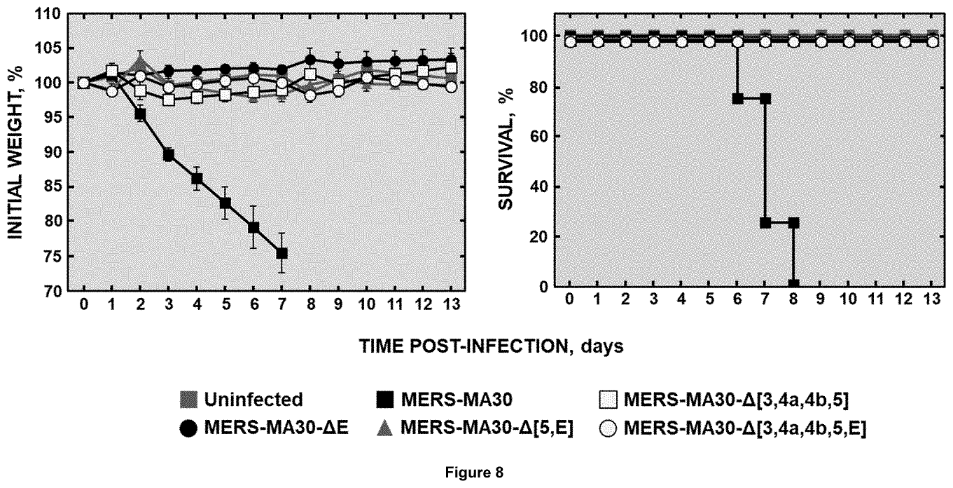

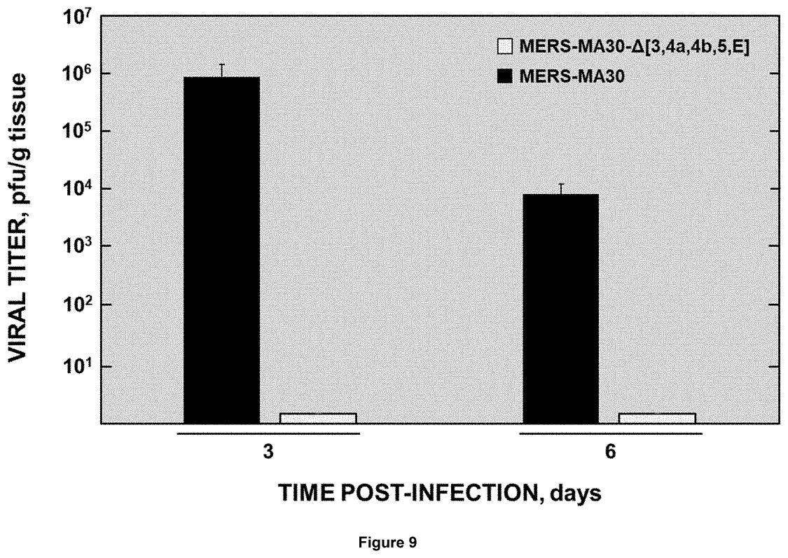

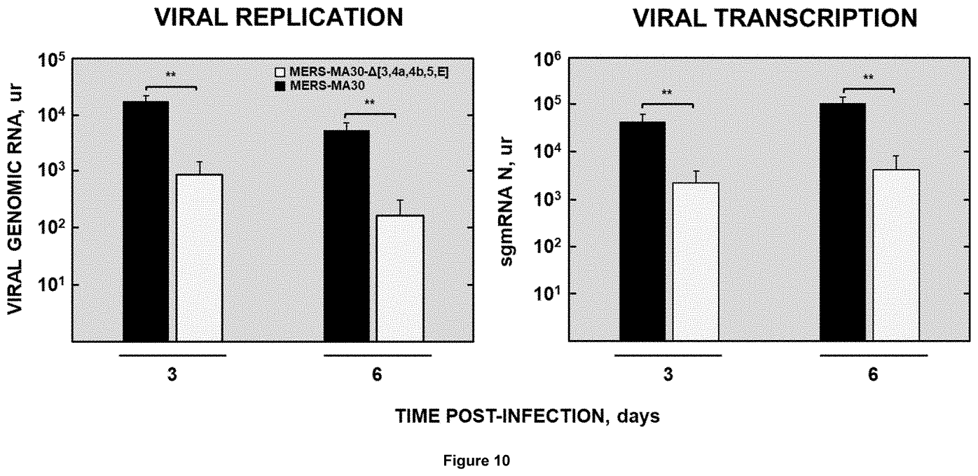

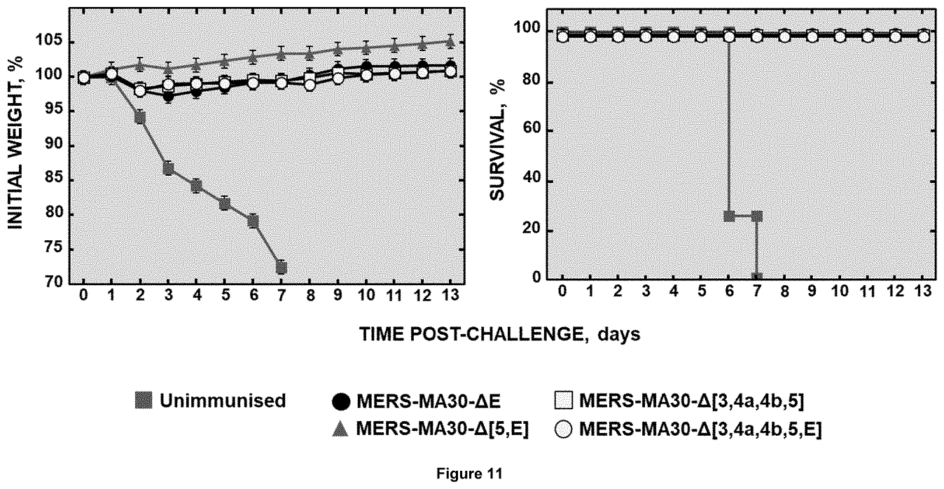

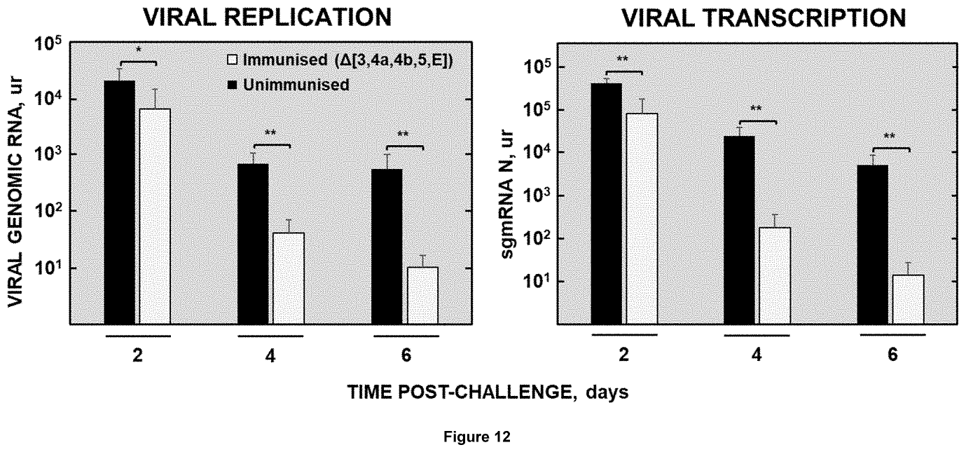

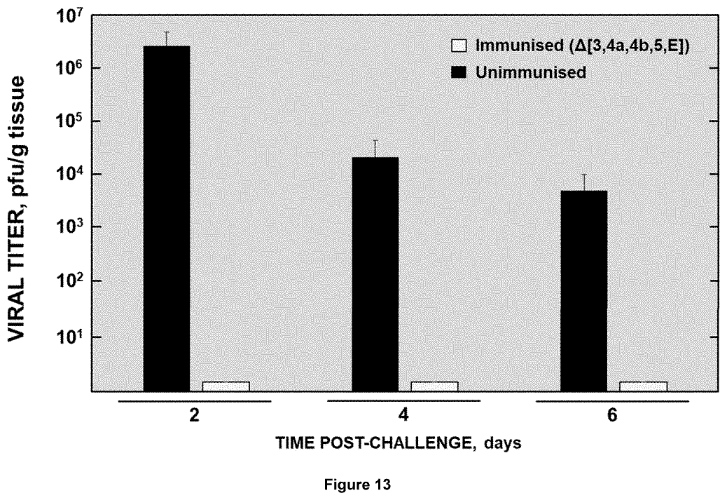

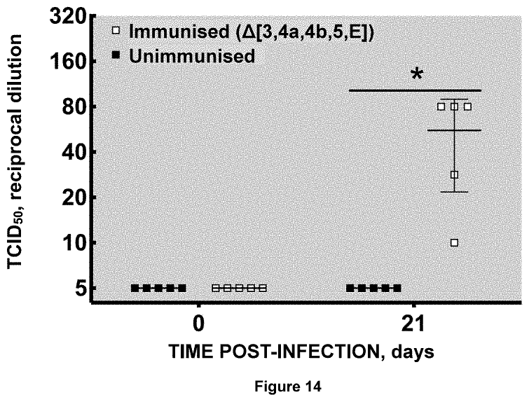

The vertical bands within the boxes represent the point mutations throughout the genome that have been introduced into the cDNA. It is important to highlight that a deletion within gene 5 (in white) and a stop codon (asterisk) have been introduced in MERS-MA30 during adaptation, which prevents the expression of protein 5 in the MERS-MA30. Black vertical lines within the boxes indicate silent mutations. (B) The upper image represents the mouse-adapted MERS-CoV genome cDNA (MERS-MA30), GenBank accession number MT576585, as shown in panel A, flanked by the cytomegalovirus (CMV) promoter and the hepatitis delta virus ribozyme (Rz) and bovine growth hormone (BGH) termination sequence. pA, poly(A) tail. The lower image represents the six fragments (F1 to F6, in dark gray) originally designed to assemble the infectious cDNA of MERS-CoV (Almazan, et al, 2013), flanked by the indicated restriction sites (the positions in the viral genome are indicated by numbers in parentheses). Above them there are light gray boxes indicating the synthetic mouse-adapted fragments chemically synthesized with the mutations mentioned in FIG. 2A (vertical black lines) (FS 1-9, Table 3). The vertical dotted lines indicate the location of each of these synthetic fragments in the infectious cDNA of MERS-CoV and in each of the fragments designed (pBAC-SA-F1-6) to assemble it.

FIG. 3. Scheme of the deletion mutants designed from the infective cDNA clone of MERS-MA30. Deleted genes are indicated in white boxes with dashed borders. There is a deletion within gene 5 (blank band and dashed border) and a stop codon (asterisk, *), which prevents expression of the entire 5 protein in MERS-MA30. The arrowheads to the left of the mutant names indicate those that, because they contain the deletion of the gene encoding protein E, are propagation defective replicons.

FIG. 4. Scheme of the MERS-CoV V1-CD and V1-VLP RNA replicons: The schemes of the V1 versions are shown, both chemically defined (CD), and packaged in VLPs (VLP). The deleted genes are indicated in white boxes with dashed borders, as well as the small deletion in the nspl gene (nsp1-ΔD), which is shown as an example.

The identity between both sequences and the modifications in the S gene of each replicon are also shown: S* opt: S gene sequence with optimised codons.

FIG. 5. Virulence of the virus obtained from the infectious cDNA of MERS-MA30. KI mice were intranasally inoculated with 10⁴ PFU/mouse of this virus isolated from mice (circles), or of the recombinant MERS-MA30 virus rescued from the infectious cDNA (black boxes). On the left figure, weight losses of infected mice are shown, and on the right graph, survival of mice over time is represented. Differences in weight loss are represented as the mean±standard error of the mean.

FIG. 6. Growth kinetics of viruses and replicons derived from MERS-MA30. Growth in the absence (E−) or in the presence (E+) of the E protein provided in trans. Huh-7 cells were infected at a MOI of 0.001 with the indicated viruses or replicons and infection was followed for 72 hours. The results are expressed as the mean±standard deviation.

FIG. 7. Transmission electron microscopy of Huh-7 cells infected with MERS-CoV-WT virus or MERS-CoV-ΔE replicon, in the absence of protein E provided in trans. Infections were made at two different multiplicities of infection (MOI), 0.1 and 1.0, with the same results, except for a greater cytopathic effect (lower cell integrity) observed in cells infected at MOI 1. Only MOI 1.0 infection is shown. Samples were taken at 17 hours after infection. On the left panel (MERS-CoV-WT), large vesicles with a high concentration of spherical virions can be observed. Vesicles with virions inside were elongated in MERS-CoV-ΔE infection (right panel) at both MOls. MERS-CoV-ΔE infected cells showed a lower cytopathic effect than those infected with MERS-CoV-WT.

FIG. 8. Evaluation of the attenuation of mutants and replicons derived from MERS-MA30 in KI mice. Weight losses of infected mice are shown on the left figure, while survival of mice is shown on the right graph. Weight losses are represented as the mean±standard error of the mean.

FIG. 9. Titer of MERS-MA30 virus and MERS-MA30-Δ[3,4a,4b,5,E] replicon in the lungs of infected mice. Titers of MERS-CoV-MA and MERS-MA30-Δ[3,4a,4b,5,E] replicon in the lungs of mice are shown. Virus titers in mice infected with MERS-CoV-MA (black columns) were at least four log units higher than those of MERS-MA30-Δ[3,4a,4b,5,E] (clear columns). Furthermore, no infectious virus was detected in mice inoculated with the replicon MERS-MA30-Δ[3,4a,4b,5,E], confirming that it was propagation deficient.

FIG. 10. Replication and transcription levels of the MERS-MA30 virus and the MERS-MA30-Δ[3,4a,4b,5,E] replicon in the lung of infected mice. Left figure shows the replication levels of MERS-MA30 virus (black columns) and MERS-MA30-Δ[3,4a,4b,5,E] replicon (light columns) in mice infected at the indicated times post infection. Right figure shows transcription levels of MERS-MA30 virus (black columns) and MERS-MA30-Δ[3,4a,4b,5,E] replicon (light columns) in the lungs of mice. **: Student's t-test (p value<significance level of 0.01); results are expressed as mean±standard deviation.

FIG. 11. Evaluation of the protection conferred by mutants and replicons derived from MERS-MA30 in KI mice. Left figure shows weight losses of mice immunised with the indicated replicons and then challenged with a lethal dose of the virulent virus. Survival of mice after the challenge is shown and on the right graph. Weight losses are represented as the mean±standard error of the mean.

FIG. 12. Replication and transcription levels of the challenge virus MERS-MA30 in the lung of mice immunised with the MERS-MA30-Δ[3,4a,4b,5,E] replicon. Left figure shows the replication level of the MERS-MA30 virus used in the challenge, in non-immunised mice (black columns) and in those immunised with the replicon (light columns). Right figure shows the transcription level of the challenge virus in the lungs of mice. Color codes are analogous to the left panel. t-Student test: (*) significance level less than 0.05; (**) significance level less than 0.01; results are expressed as mean±standard deviation.

FIG. 13. Titers of challenge virus in the lungs of mice immunised with the MERS-MA30-Δ[3,4a,4b,5,E] replicon. Black columns show viral titers in non-immunised mice, while light columns show titers in mice immunised with the replicon. It was clearly observed that in immunized mice, no detectable levels of challenge virus were found at any time after immunisation, indicating that immunization with the replicon provided sterilizing immunity, i.e. that the challenge virus could not grow in the lungs at any time.

FIG. 14. Amount of neutralizing antibodies in the serum of mice immunized with the replicon. Blood samples were obtained from mice immunised with the MERS-MA30-Δ[3,4a,4b,5,E] replicon or control (non-immunised) mice at 0 and 21 days after infection. Neutralizing antibody titer is shown as the highest serum dilution showing complete neutralization of the cytopathic effect in 50% of the wells (TCID50). Student's t-test: (*) p value: 0.0102919<0.05.

### EXAMPLES

Materials and Methods

The materials and protocols common to the different examples are described below and will be those used unless explicitly stated otherwise.

Eukaryotic Cell Lines

The following cell lines were used: Huh-7 cells derived from human (Homo sapiens) hepatocarcinoma, BHK21 cells derived from newborn golden hamster (Mesocricetus auratus) kidney, Vero-81 and Vero E6 cells derived from green cercopithecus (Chlorocebus aethiops) kidney, human embryonic kidney HEK293 cells, human lung adenocarcinoma-derived Calu3 and Calu3 2B4 cells, and human lung fibroblast-derived MRC-5 cells.

Cell lines were grown in an incubator at 37° C. with 5% CO₂ partial pressure and 97% humidity in Dulbecco's modified Eagle medium (DMEM) with 25 mM HEPES [4-(2-hydroxyethyl)piperazin-1-ylethanesulfonic acid] buffer and 4.5 g/L glucose (BioWhittaker, Lonza). For cell culture assays, the medium was supplemented with 2 mM glutamine (Sigma-Aldrich), 1% p/v non-essential amino-acids (Sigma-Aldrich) and 10% v/v FBS. 100 IU/mL gentamicin (Sigma-Aldrich) was added to the medium for line maintenance between assays. All cell lines were cryopreserved in liquid nitrogen at a density of 1×10⁶ cells/mL in FBS with 10% v/v dimethyl sulfoxide (DMSO) (Sigma-Aldrich).

Bacteria

For the cloning of the different plasmids, Escherichia coli strain DH10B (Invitrogen, Thermo Fisher Scientific) was used, this strain has the following phenotype:

[F-mcrAΔ(mrr-hsdRMS-mcrBC)ø80dlacZΔM15ΔlacX74deoRrecA1endA1araD139 (ara,leu)7697galUgalKλ-rpsLnupG]

Bacteria were grown at 30 or 37° C. in Luria-Bertani (LB) liquid medium (Sambrook and Russell, 2001), or in LB-agar solid medium (15 g/L) for colony isolation. When necessary, the medium was supplemented with antibiotics for the selection and growth of individual colonies (100 μg/mL ampicillin or 12.5 μg/mL chloramphenicol; Sigma-Aldrich).

Generation of Competent Bacteria

For the preparation of DH10B bacteria competent for electroporation, a pre-inoculum was grown at 37° C. overnight from an individual colony isolated on solid LB-agar medium, in 50 mL of Super Optimal Broth (SOB) medium [20 g/L tryptone (Becton, Dickinson and Company), 5 g/L yeast extract (Becton, Dickinson and Company), 0.5 g/L NaCl (Sigma-Aldrich), 0.18 g/L KCI (Sigma-Aldrich)]. The next day, two liters of SOB medium were inoculated with 1 mL of the pre-inoculum and amplified to an optical density at 550 nm (OD_{550 nm}) of 0.7. They were then sedimented by centrifugation at 4000×g for 10 min at 4° C. and washed three times with a 10 (Yov/v glycerol solution. In the first wash the bacteria were resuspended in a volume equivalent to that of the initial culture, which was halved in successive washes (2 L, 1 L, 0.5 L and 0.25 L, respectively). After initial sedimentation and between washes, the bacteria were kept on ice at all times. The final sediment was resuspended in a volume of 6 mL of 10% v/v glycerol at 4° C., the bacteria were divided into aliquots and frozen at −80° C. until use.

Transformation of Bacteria by Electroporation

Electroporation-competent DH10B bacteria were transformed with a salt-free DNA. That DNA was dialyzed for 20 min against distilled H₂O using hydrophilic cellulose ester membranes with a pore size of 0.025 μm (Merck-Millipore). Dialyzed DNA was mixed with 50 μL of competent bacteria and transferred to a 0.2-cm electroporation cuvette (Bio-Rad). For electroporation, an electric pulse of 25 μF, 2.5 KV and 200Ω was applied to the cuvette with the DNA-bacteria mixture with a MicroPulser Electroporator electroporator (Bio-Rad). The electroporated cells were then resuspended in 1 mL of LB and grown for 45 min at 37° C. under shaking. After incubation, the electroporated bacteria were seeded on a plate of LB-agar medium with the corresponding selection antibiotic.

DNA Manipulation and Analysis

Plasmids

The plasmid TRE-Auto-rtTA-V10-2T was used for the expression of the envelope protein (E) of MERS-CoV and its variants. The sequence of the resulting constructs was verified by Sanger sequencing (Macrogen).

The gene of interest was under the influence of an inducible promoter in the plasmid TRE-Auto-rtTA-V10-2T (Das et al., 2016b). This vector was based on the tetracycline-controlled inducible expression system Tet-On (Das et al., 2016a). Two EcoRl restriction sites were introduced by PCR to clone the gene encoding the E protein or its variants into the plasmid. Oligonucleotides used for cloning were: VS-EcoRI-E-MERS-rtTA-V10-2T (5′-CCGGAATTCGAGCTCGGT ACCCGGGGATCCACCGGTCGCCACCATGTTACCCTTTGTC-3′) SEQ_ID 22 and RS-EcoRI-EMERS-rtTA-V10-2T, SEQ_ID 23. The resulting PCR product was cloned into the vector using the EcoRI restriction sites. The correct orientation of the insert in the vector was checked by PCR using an internal (RS-E-MERS: 5′-TTAAACCCACTCGTCAGG-3′) SEQ_ID 24 and an external (VS-TRE-Auto-2380: 5′-ATCCACGCTGTTTTGACCTC-3′) SEQ_ID 25 oligonucleotide with respect to the inserted fragment. The TRE-Auto-rtTA-V10-2T plasmid contained a gene encoding a transactivator (rtTA) downstream of the gene encoding the E protein. In the presence of an inducer (doxycycline, a tetracycline-derived antibiotic), this transactivator was able to bind to the inducible promoter and initiate transcription of the gene of interest. Between the two genes there was an Internal Ribosome Entry Site (IRES), which generated a positive feedback loop that increased the expression levels of both the E protein and the transactivator.

The high copy number plasmid pUC57 (GenScript) was used for cloning and modification of some DNAs complementary to the viral RNA (cDNA). To clone the cDNA of MERS-CoV strain EMC/2012, MERS-MA30, and its corresponding variants, the plasmid pBeloBAC11 (pBAC) (Wang et al., 1997) was used. This 7507 bp plasmid contains the E. coli factor F origin of replication (oirS), the chloramphenicol resistance gene (cat) and the genes necessary to maintain a single copy of the plasmid per cell (parA, parB, parC and repE). This vector was also used for cloning and modification of large viral cDNAs or those containing toxic sequences for bacteria, as previously described (Almazán et al., 2000; Gonzalez et al., 2002).

Plasmid and DNA Fragment Purification

For the extraction and purification of plasmids from small- to medium-scale bacterial cultures, the Plasmid Mini Kit and Plasmid Midi Kit (Qiagen) reagents were used, respectively.

For large-scale purification of pBAC-based plasmids, a 400 mL bacterial culture grown for 18 h at 30° C. under agitation in LB medium supplemented with 15 μg/mL chloramphenicol (Sigma-Aldrich) was used. The Large Construct Kit reagent (Qiagen) was used for purification. This reagent allows purification of large DNA fragments free of bacterial chromosomal DNA by an exonuclease treatment.

PCR products and DNA fragments extracted from agarose gels were purified with the QIAquick Gel Extraction Kit reagent (Qiagen). When the fragment was larger than 10 kb, QIAEX II reagent (Qiagen) was used.

In all cases, the manufacturer's instructions were followed and DNA was eluted in ultrapure distilled H₂O MilliQ (Merck-Millipore) or EB elution buffer (10 mM Tris-Cl, pH 8.5; Qiagen).

Restriction Enzymes, DNA Modification and DNA Ligation.

All restriction enzymes used in cloning and restriction patterning were obtained from Roche, New England Biolabs, and Thermo Fisher Scientific. For DNA ligation reactions, the DNA ligase enzyme from phage T4 (Roche) was used. Dephosphorylation of DNA molecules ends was performed with shrimp alkaline phosphatase (Applied Biosystems, Thermo Fisher Scientific). Restriction enzyme treatments, dephosphorylation and DNA ligation were carried out following each manufacturer's instructions and standard protocols previously described (Sambrook and Russell, 2001). The sequence of the resulting constructs was analyzed and verified by Sanger sequencing (Macrogen).

Polymerase Chain Reaction for the Amplification of DNA Fragments.

Amplification reactions were performed on a 2720 Thermal cycler or a SimpliAMP thermal cycler (Applied Biosystems, Thermo Fisher Scientific). The final volume of the reactions was 25 μL. In those preparative reactions in which it was necessary to obtain a larger amount of PCR product, the final volume was increased to 50 μL. Between 50 and 150 ng of template DNA were used per reaction. The melting temperature of the oligonucleotides (Tm) and the length of the fragment to be amplified determined the hybridization temperature (4 to 5° C. less than the Tm of the oligonucleotide with lower Tm) and the elongation time (about 1 min per 1 kb of amplified DNA), respectively. The reaction conditions were adjusted as follows: (a) initial denaturation of 5 minutes at 95° C.; (b) 25-35 cycles of: i) denaturation, 30 seconds at 95° C.; ii) hybridization, 30 seconds at the calculated temperature; iii) elongation, 1 minute/kb at 72° C.; (c) final elongation, 10 minutes at 72° C.

Reactions for analytical purposes, including genotyping, were carried out with AmpliTaq DNA polymerase enzyme (Applied Biosystems, Thermo Fisher Scientific). 0.025 U/μL of polymerase in its corresponding reaction buffer (GeneAmp 10× PCR Buffer II, Applied Biosystems, Thermo Fisher Scientific) in the presence of 2.5 mM MgCl₂, 0.3 μM of each oligonucleotide and a mixture of deoxynucleotide triphosphates (dNTPs) (Roche) at a final concentration of 0.2 mM of each were used.

For preparative and sequencing reactions, Vent polymerase enzyme (New England Biolabs) was used, which exhibits higher fidelity due to its error-correcting 3′-5′ exonuclease activity. 0.016 U/μL of polymerase in its corresponding reaction buffer (ThermoPol Reaction Buffer, New England Biolabs; final composition 1×: Tris-HCl 20 mM, (NH₄)₂SO₄ 10 mM, KCl 10 mM, Triton® X-100 0.1%, pH 8. 8) in the presence of 2 mM MgSO₄, 0.2 μM of each oligonucleotide and a dNTPs mixture at a final concentration of 0.3 mM of each.

DNA Electrophoresis on Agarose Gels.

Separation of DNA fragments for analytical studies or purification was performed by electrophoresis on 0.7-1.5% wt/v low-EGD agarose gels (Pronadisa, CONDA Laboratories) in TAE buffer (40 mM Tris-acetate, 1 mM ethylenediaminetetraacetic acid—EDTA—1 mM). For visualization of DNA bands on a ChemiDoc imager (Bio-Rad) SYBR Safe DNA gel stain 1× (Invitrogen, Thermo Fisher Scientific) was incorporated into the agarose solution.

Viruses

### Viral Isolates

Recombinant viruses rescued from transfection of the MERS-CoV infectious clone (MERS-CoV) (Almazán et al., 2013) have the genetic background of the MERS-CoV isolate EMC/2012 (GenBank: JX869059) (van Boheemen et al., 2012). Recombinant viruses rescued from transfection of the mouse-adapted MERS-CoV infectious clone (MERS-MA30) exhibit the genetic background of the MERS-CoV-6-1-2 isolate after 30 passages in hDPP4-knockin mice (Li et al., 2017).

Virus Manipulation in Cell Culture.

Viruses were grown in cells following standard protocols. For this purpose, cells were grown to 100% confluence in preferably screw-capped culture flasks or culture plates. They were then brought to the NCB3 laboratory and infected with the desired amount of virus. In the case of culture plates, they were placed in heat-sealable plastic bags. Both flasks and plates were placed in methacrylate boxes for spill containment and incubated at 37° C. for the indicated period of time.

Batches of virus were generated in screw-capped culture flasks of the desired final batch volume. At 24 hours after seeding the cells and verifying that they had reached 100% confluence, they were infected at a multiplicity of infection (MOI) of 0.001 plaque-forming units (PFU) per cell (PFU/cell). The supernatant was collected 72 hours post infection (hpi) and distributed into aliquots that were stored at −80° C. until use. The sequence of the virus batches was analyzed by Sanger sequencing (Macrogen) to verify that no changes had occurred.

Viral Titration by Plaque-Forming Assay

Titrations by plaque-forming assay were carried out following standard protocols adapted to the virus strains used in the laboratory (Coleman & Frieman, 2015). Twelve-well plates were seeded with Huh-7 or Vero 81 cells, grown to 100% confluence and infected in triplicate with factor-10 serial dilutions of the virus supernatant. After 45 min of adsorption at 37° C., the medium was removed and DMEM supplemented with 4 mM glutamine, 1%v/v non-essential amino acids, 2%v/v FBS, 0.16 mg/mL DEAE-Dextran and 0.6% p/v low-electroendoosmosis point agarose (Pronadisa, CONDA Laboratories) was added, forming an agarose layer. Huh-7 or Vero E6 and Vero81 cells were infected with MERS-CoV and incubated for 72-96 hours. After incubation, the cells were fixed and inactivated with 10% v/v formaldehyde (Sigma-Aldrich) in phosphate buffered saline (PBS) for at least 45 min at room temperature. The formaldehyde and agarose plug were then removed to stain the cells with a crystal violet solution (1 mg/mL crystal violet in 20% methanol in distilled H₂O) for 15 min at room temperature. The number of lysis plates formed in each of the dilutions was determined. The titer was expressed as the number of PFU multiplied by the dilution factor in a volume of 1 mL (PFU/mL).

Viral Titration by Immunofluorescence Focus Formation Detection Assay

The immunofluorescence focus formation detection assay is particularly useful for the detection and titration of those spreading-deficient viruses unable to form visible plaques. For this purpose, 5×10⁴ cells per well were seeded in a 96-well plate in a final volume of 50 μL of medium. The next day, cells were infected with 20 μL of factor-10 serial dilutions of the virus-containing supernatant. At 16 h post infection (hpi), cells were fixed and inactivated with 4% p/v paraformaldehyde (Merck-Millipore) in PBS for 45 min at room temperature, washed with PBS, and permeabilized with cold methanol for 20 min at room temperature. Nonspecific binding sites were blocked with 10% v/v FBS in PBS for one hour at room temperature. Cells were then incubated overnight at 4° C. with a rabbit polyclonal antibody against the nucleocapsid (N) protein (BioGenes) at a 1:500 dilution in 5% FBS in PBS. The next day, the antibody was removed, cells were washed with PBS and incubated with a goat anti-rabbit monoclonal antibody conjugated with Alexa Fluor®488 fluorochrome (Invitrogen, Thermo Fisher Scientific) at a 1:500 dilution in 5% FBS in PBS for 45 minutes at room temperature. Infection foci formed at the different dilutions were counted using an Axio Vert.A1 fluorescence microscope (Zeiss). The titer was expressed as the number of focus-forming units (FUs) multiplied by the dilution factor in a volume of 1 mL (FUs/mL), equivalent to PFU/mL.

Viral Titration by the 50% Tissue Culture Infective Dose Method

The 50% tissue culture infective dose (TCID₅₀) is the dilution at which the virus produces a cytopathic effect in 50% of the wells with inoculated cells. To perform titration by this method, 5×10⁴ cells per well were seeded in a 96-well plate in a final volume of 50 μL of medium. The next day, the medium was removed and the cells were infected with 100 μL of factor-10 serial dilutions of the virus supernatant (from 1:10 to 1:10⁸) and incubated for 72 hours at 37° C. For each of the dilutions of each virus, 10 wells were inoculated. At 72 hpi, the medium was removed from the cells and they were fixed and inactivated with 10% v/v formaldehyde (Sigma-Aldrich) in PBS for at least 45 min at room temperature. The formaldehyde was then removed to stain the cells with a crystal violet solution (1 mg/mL crystal violet in 20% methanol in distilled H2O) for 15 min at room temperature. To obtain the titer, the dilution of virus at which 50% of the wells with cells showed cytopathic effect (TCID₅₀) was calculated following the method described by Reed-Muench (Reed and Muench, 1938), multiplied by the dilution factor and expressed as TCID₅₀ per milliliter of virus (TCID_{50/mL}).

Transfection of Infective cDNAs and Rescue of MERS-CoV and MERS-MA30 Viruses.

BHK21 cells grown to 95% confluence in 12.5 cm² culture flasks were transfected with 6 μg of the infective cDNA of one of the viruses and 18 μL of the transfection reagent Lipofectamine 2000 (Invitrogen, Thermo Fisher Scientific), according to the manufacturer's specifications. At 5-6 hours after transfection (hdt), transfected BHK21 cells were detached from the plate with 500 μL of trypsin-EDTA (25%), added onto a monolayer of confluent Huh-7 or Vero 81 cells grown in 12.5 cm² culture flasks and incubated at 37° C. After 72 hours, supernatants were collected (passage 0) and stored at −80° C. One third of the supernatant was reserved to make a passage to a new flask of confluent Huh-7 or Vero 81 cells, which were incubated, again, at 37° C. for 72 hours. After incubation, the supernatants (passage 1) were collected and stored at −80° C. The rescued viruses were amplified directly from passage 1. Only selected viruses were cloned prior to amplification by three lysis plate purification steps in semi-solid DMEM medium at 0.6% w/v of low electroendoosmosis point agarose (Pronadisa, CONDA Laboratories). Passages 0 and 1, as well as the amplification batches of each virus, were titered and sequenced to verify that rescue had occurred correctly.

Rescue of MERS-CoV Derived Replicons

For the rescue of MERS-CoV replicons with the gene encoding the E protein deleted, Huh-7 cells were transfected with the plasmid pcDNA3.1-E-MERS-CoV. This plasmid was used to provide in trans the E protein in order for the replicon to form VLPs carrying the E protein on its surface. In this way, the generated replicon was self-sufficient to infect on its own. Alternatively, to rescue the replicon within VLPs, Huh-7 cells were cotransfected with the MERS-CoV replicons and the TRE-Auto-rtTA-V10-2T-E-MERS-CoV plasmid that provides the E protein in trans. At 5-6 hpt, the medium with the plasmid-Lipofectamine complexes was removed from the transfected Huh-7 cells, cells were washed, and fresh medium was added. For cells transfected with the TRE-Auto-rtTA-V10-2T-EMERS-CoV plasmid, the medium was supplemented with doxycycline at a concentration of 1 μg/mL to induce E protein expression. In an alternative process, the same plasmids were first transfected onto BHK21 cells, and after 6 hours cells were detached from the plate, by incubating with 500 μL of trypsin-EDTA (25%), and added onto Huh-7 cells transfected with the E protein expression plasmids and incubated at 37° C. for 72 hours. The prior transfection of the plasmids on BHK21 cells was done to increase transfection efficiency, since a high percentage of BHK21 transfected cells was obtained. For both rescue and successive amplification passages and batch generation of the viruses, Huh-7 cells were transfected with the E protein expression plasmids at a DNA:Lipofectamine 2000 ratio of 1:3 (micrograms:microliters).

### Transmission Electron Microscopy

Huh-7 cells were seeded in 24-well plates. The next day, the confluence level was checked to be almost 100%. At that time, the cells were infected with 0.1 and 1.0 MOls of the viruses and MERS-CoV and MERS-CoV-ΔE, respectively, obtaining similar results in both cases. At 17 hpi, the medium was removed, several washes were made with PBS buffer, and cells were fixed in situ for 2 h at room temperature with a solution of 4% w/v PFA and 2% w/v glutaraldehyde in 0.1 M Sörensen's phosphate buffer at pH 7.4. They were stored at 4° C. for 24 h for fixation. Inclusion of the cells was done directly on the plate in plane, without detaching the cells. For this purpose, the fixative was removed and the cells were embedded in TAAB 812 epoxy resin (TAAB Laboratories). The resin blocks were removed from the plate for ultrathin cuts (70-80 nm) with an Ultracut E ultramicrotome (Leica) that were counterstained with a solution of 2% uranyl acetate in water and Reynolds lead citrate. The grids with the slices were examined at 80 kV in a JEM1010 transmission electron microscope (Jeol) and pictures were taken with a CMOS TemCam F416 digital camera (TVIPS).

RNA Manipulation and Analysis

Extraction and Purification of Intracellular Total RNA

Total RNA from infected mouse cells or lungs was extracted and purified with the RNeasy Mini Kit reagent (Qiagen) for sequence verification and stability analysis of the rescued viruses, as well as for quantification of viral and cellular gene expression. The purification yield was quantified with a NanoDrop ND-1000 spectrophotometer (NanoDrop Technologies). All purified RNAs were stored at −80° C. until use.

Synthesis of cDNAs from RNA by RT-PCR

The cDNAs were synthesized from purified RNAs by reverse transcription (RT)-PCR with the High Capacity DNA RT reagent kit (Applied Biosystems, Thermo Fisher Scientific) in a final volume of 30 μL, with 150 ng of RNA as template and random hexanucleotides, provided in the kit, as primers. RT-PCR conditions were: 10 minutes at 25° C., 120 minutes at 37° C. and 5 minutes at 85° C. for enzyme inactivation. The cDNAs generated were used immediately and the remainder was stored at −20° C. A fraction of the cDNAs (2 μL) was used as a template for PCR amplification using specific oligonucleotides. The products of this PCR were analyzed by agarose gel electrophoresis and Sanger sequencing (Macrogen) to study the stability and sequence of the mutants generated.

Quantification of RNAs by Quantitative RT-PCR (RT-qPCR)

Using TaqMan technology, viral genomic (gRNA) and subgenomic (sgmRNA) RNA present in mouse lung samples was transcribed to cDNA by reverse transcription and analyzed by quantitative PCR (RT-qPCR). For gRNA and N-gene sgmRNA (sgmRNA-N), TaqMan assays were composed of two oligonucleotides (Sigma-Aldrich) and a fluorophore-conjugated probe with a fluorescence deactivator (Eurofins Genomics). Both the oligonucleotides and the probe were specific for MERS-CoV and MERS-MA30 viruses (Table 2).

TABLE 2
TaqMan assays.

| Probe | Sequence 5′→3′ probe | Oligonucleotides | Sequence 5′→3′ probes |
|---|---|---|---|
| gRNA- | TGCTCCAACAGTTACAC | VS MERS gARN | GCACATCTGTGGTTCTCCTCTCT |
| MERS | | SEQ_ID 28 | |
| SEQ_ID | | RS MERS gARN | AAGCCCAGGCCCTACTATTAGC |
| 26 | | SEQ_ID 29 | |
| sgmRNA- | CTTTGATTTTAACGAATC | Leader sgARN | CTTCCCCTCGTTCTCTTGCA |
| N-MERS | | SEQ_ID 30 | |
| SEQ_ID | | sgARN-N | TCATTGTTATCGGCAAAGGAAA |
| 27 | | SEQ_ID 31 | |

To normalize and viral RNA quantifications, a commercial mouse 18-S ribosomal RNA-18-S (rRNA-18S) TaqMan assay (reference: Mm03928990-g1; Applied Biosystems, Thermo Fisher Scientific) was used as an internal control.

We took 2 μL of a 1/10 dilution of the cDNA synthesized by reverse transcription for quantification of viral RNAs, and 2 μL of a 1/100 dilution for quantification of 18S ribosomal RNA (rRNA-18S). The qPCR was performed on a 7500 Real Time PCR System (Applied Biosystems, Thermo Fisher Scientific) using the following conditions: (a) 2 minutes at 50° C.; 10 minutes at 95° C.; (b) 40 cycles of: (i) 15 seconds at 95° C.; (ii) 1 minute at 60° C. In all cases the reaction was carried out with GoTaq qPCR Master Mix reagent (Promega) and three biological replicates and three technical replicates of the above were analyzed to ensure the precision of the analysis. Values corresponding to the means of the cut-off cycle (Ct) were analyzed with 7500 software v2.0.6. (Applied Biosystems, Thermo Fisher Scientific) and used to calculate relative expression values using the 2^{-ΔΔCt} method (Livak and Schmittgen, 2001).

Mouse Assays

Experimental Mouse Models and Inoculation Protocols.

For the evaluation of MERS-MA30 pathogenesis and MERS-MA30 replicon-based vaccine candidates, C57BL/6NTac-Dpp4^{tm3600(DPP4)Arte} knock-in mice were used (Li et al., 2017) (KI mice), in which exons 10-12 of the murine Dipeptidyl peptidase 4 gene (Dpp4, Cd26 or mDpp4) were replaced with the corresponding exons of the human homologous gene, DPP4, thus generating a humanized chimeric protein (huDpp4). The substituted exons encode the RBD-recognized region of the MERS-CoV S protein. These mice are very useful for the study of the pathology produced by M ERS-CoV, since they reproduce very well the clinical signs and lung damage observed in human MERS-CoV infection, (Li et al., 2017). SJL-Tg(K18-DPP4) (K18) transgenic mice were also used (Li et al., 2016) in the protection evaluations with similar results to the experiments performed on KI mice, described above for immunizations.

Mice experiments were performed on 16- to 30-week-old females free of specific pathogens. In all the experimental procedures performed, mice were anesthetized with a mixture of isoflurane and oxygen. All viruses were inoculated intranasally in a maximum final volume of 50 μL of DMEM by depositing small droplets of the virus suspension into the nostrils, which the mouse inhaled naturally within 2 to 3 seconds. Depending on each experiment, the dose used varied between, 1×10⁴ PFU for immunizations or attenuation studies and 1×10⁵ PFU/mouse for challenge.

Disease Monitoring, Virulence Analysis and Sample Collection

Infected mice were monitored for weight, clinical signs of disease and survival for a period of 14 days. Those animals that during the course of the experiment suffered weight losses greater than 25% of the initial weight were sacrificed according to the established endpoint criteria.

At the indicated days, three mice from each experimental group were sacrificed by cervical dislocation for lung sampling. For viral load counting in the lungs of infected mice, half of the right lung was taken and stored at −80° C. until use. The rest was embedded in RNAlater preservation solution (Sigma-Aldrich) for 48 hours at 4° C. and stored at −80° C. until further processing to ensure the integrity of the RNA molecules. The left lung was fixed in 10% zinc formalin solution (Sigma-Aldrich) for 24-48 hours at 4° C. for virus inactivation and subsequent histopathological analysis.

Sample Processing for Lung Virus Quantification

Lung samples were thawed and homogenized in 2 mL of PBS supplemented with 50 μg/mL gentamicin (Sigma-Aldrich), 0.25 μg/mL amphotericin B (Gibco, Thermo Fisher Scientific), 100 IU/mL penicillin (Sigma-Aldrich) and 100 μg/mL streptomycin (Sigma-Aldrich) in a gentleMACS Dissociator homogenizer (Miltenyi Biotec) with the corresponding tubes, following the manufacturer's instructions. Samples were centrifuged at 3000×g for 10 min at 4° C. Supernatants were divided into aliquots and stored at −80° C. until virus titration by the methods described above. Titers were expressed as plaque-forming units per gram of lung (PFU/g).

Lung RNA Extraction

The RNA preservation solution was removed from the lung samples and homogenised in 2 mL of RLT lysis buffer (Qiagen) with 1% v/v β-mercaptoethanol in a gentleMACS Dissociatorhomogeniser (Miltenyi Biotec), using the appropriate tubes and following the manufacturer's instructions. The homogenised samples were centrifuged at 3000×g for 10 minutes at 4° C. Total RNA was purified from the supernatant using the RNeasy Mini Kit reagent (Qiagen). The purified total RNA was used to quantify viral and cellular RNAs.

Virulence Assessment of Vaccine Candidates

For attenuation assessment of the vaccine candidates, 11 mice were infected with each virus generated: five for disease monitoring, three for lung sampling at 3 days post infection (dpi), and three for lung sampling at 6 dpi. KI mice were inoculated with 1×10⁴ PFU/mouse of the replication-competent propagation-defective virus based candidates.

As a control during disease follow-up, five K18 mice were inoculated with 5×10³ PFU/mouse of MERS-CoV and five KI mice with 1×10⁴ PFU/mouse of MERS-MA30.

Assessment of Protection of Vaccine Candidates

At 21 days post-immunisation (dpim), the five mice immunised with each vaccine candidate were challenged with a high dose (1×10⁵ PFU/mouse) of MERS-CoV or MERS-MA30, depending on whether they were K18 or KI mice, respectively. After assessment of the degree of protection, one candidate of each vaccine type was selected to immunise 12 mice and samples were taken at days 2, 4, 6, 8 and 12 days post-challenge (dpc). The collected samples were used to measure viral RNA levels, viral load and to assess the ability of the selected candidates to induce sterilising immunity.

Virus Neutralisation Assay

Blood samples were obtained from the submandibular vein at 0 and 21 days after immunisation. Blood samples were incubated at 37° C. for 1 hr in a water bath and then at 4° C. overnight to facilitate coagulation and separation of serum. The serum was clarified by centrifugation and stored at −80° C. One day before the assay, 5×10⁴ Huh-7 cells per well were seeded in 96-well plates. On the day of the assay, serum samples were thawed and incubated at 56° C. for 30 min to inactivate complement. Twofold dilutions of each serum were prepared in complete DMEM supplemented with 2% FBS in a final volume of 60 μL. Serum dilutions were incubated for 1 hr at 37° C. with 100 TCID₅₀ of MERS-MA 30 in a 1:1 proportion. Medium was removed from Huh-7 cells, and cells were incubated with 60 μL of serum:virus mixtures for 1 hr at 37° C. After incubation, the serum:virus mixture was replaced with fresh complete DMEM medium, and cells were incubated at 37° C. for 72 hr. Finally, cells were fixed with 10% v/v formaldehyde in PBS and stained with crystal violet. The titer of neutralising antibodies in mouse serum was determined as the highest dilution showing complete neutralisation of the cytopathic effect in 50% of the wells (TCID₅₀).

Statistical Analysis

To analyse the differences between the means of two groups, the two-tailed Student's t-test for unpaired samples was used. For the comparison of means of three or more groups, one-way analysis of variance (ANOVA) was used. P values<0.05 were considered significant. Differences in weight loss were represented as the mean±standard error of the mean (SEM). All other results were expressed as the mean±standard deviation (SD).

### EXAMPLES

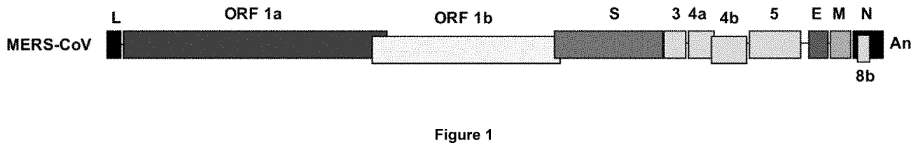

As it was stated above, MERS-Cov is one of the deadliest coronavirus for humans, FIG. 1 shows a schematic representation of its complete genome.

Several examples of the method of the invention by which MERS-CoV derived RNA replicons are obtained and their use as vaccines for the generation of immunity in animal models are described below.

1. Engineering of Mouse-Adapted MERS-CoV Infective Clones Isolated after 30 Passages in hDPP4-Knockin Mice (MERS-MA30)

The use of coronavirus-derived RNA replicons as vaccines requires the use of animal models. In this first example, mice (Mus musculus) are not susceptible to MERS-CoV infection, since the S protein of MERS-CoV does not recognise the murine homologous protein of the human receptor. For this reason, two mouse models genetically modified to be susceptible to MERS-CoV infection have been used and a derivative of MERS-CoV that is pathogenic in these animals has been used.

From the MERS-CoV Genbank JX869059 virus sequence, a mouse-adapted strain was generated, causing the death of all infected mice, by passing the MERS-CoV virus for 30 consecutive times in mice (Li et al., 2017). This virus was named MERS-MA30-6-1-2 (SEQ_ID 21). In the present invention, the full-length cDNA of the coronavirus genome described in the attached SEQ_ID 21 has been generated and deposited in GenBank under accession number MT576585. By the design of nine fragments chemically synthesised (GeneArt, Thermo Fisher Scientific) containing the mutations present in the MERS-MA30-6-1-2 virus (FIG. 2A), isolated after 30 passages in hDPP4-knockin mice (MERS-MA30). This animal model and virus were selected because, together, KI mice and MERS-MA30-6-1-2 best reproduce in the mouse the clinical signs observed in humans.

The cDNA was cloned into a BAC (Almazán et al., 2013) under the cytomegalovirus (CMV) immediate early promoter and an untranslated region (UTR) and is flanked at the 3′ end by the bovine growth hormone (BGH) termination and polyadenylation sequences separated from the poly A tail (with 24 adenine residues) by the HDV ribozyme sequence (Rz).

Each of these fragments was cloned into a pBAC intermediate plasmid (Almazán et al., 2013) using selected restriction enzymes (FIG. 2B, Table 3). The different MERS-MA30 fragments were then assembled from the pBAC intermediate plasmids, as previously described (Almazán et al., 2013), to generate the corresponding pBAC with the complete MERS-MA30 genome (pBAC-MERS-MA-FL).

TABLE 3
DNA fragments containing the mutations of
the mouse-adapted MERS-CoV, MERS-MA30.

| Synthes. | | | | | | |
|---|---|---|---|---|---|---|
| fragment | Fragment | | | Intermediate | Viral | |
| (FS) ^{(a)} | size (bp) | 5′ end ^{(b)} | 3′ end ^{(b)} | plasmid ^{(c)} | genome ^{(d)} | |
| 1 | 1424 | AscI | BamHI | pBAC-SA-F1 | 1-806 | nt |
| 2 | 1725 | PfI23II | Pfl23II | pBAC-SA-F2 | 807-7622 | nt |
| 3 | 918 | NsiI | NsiI | pBAC-SA-F4 | 9075-20901 | nt |
| 4 | 2915 | Bsu36I | Bsu36I | pBAC-SA-F4 | 9075-20901 | nt |
| 5 | 1488 | SphI | SphI | pBAC-SA-F4 | 9075-20901 | nt |
| 6 | 3493 | ApaLI | ApaLI | pBAC-SA-F5 | 20902-25840 | nt |
| 7 | 191 | MfeI | BgIII | pBAC-SA-F6 | 25841-30162 | nt |
| 8 | 887 | NdeI | KfII | pBAC-SA-F6 | 25841-30162 | nt |
| 9 | 2635 | KfII/SanD1 | RsrII | pBAC-SA-F6 | 25841-30162 | nt |

^{(a)} According to the scheme in FIG. 2B.
^{(b)} Restriction enzymes used to clone the synthesised fragments into the intermediate plasmids.
^{(c)} Intermediate plasmid into which the synthesised fragment has been cloned (Almazán et al., 2013).
^{(d)} Nucleotides of the viral genome included in the intermediate pBAC. nt: nucleotide. The virus genome starts at position 7890 of pBAC-MERS-MA30-FL.

In addition to the genome of the MERS-MA30-6.1.2 clone, in which the ORF5 gene is mutated and not expressed (Li et al., 2017), other infectious cDNAs were generated: one with the full ORF5 gene (pBAC-MERS-MA30-5FL) and one with the ORF5 gene deleted (pBAC-MERS-MA30-Δ5) (not shown).

2. Assessment of MERS-MA30 Virulence In Vivo

MERS-CoV, which infected KI mice without causing death, was adapted to grow in these mice by 30 sequential virus passages, resulting in the virus named MERS-MA30 (mouse adapted MERS-CoV). After this process, the virus caused the death of infected KI mice. Subsequently, it was cloned three times by lysis plaque isolation, and a clone was selected for further work,named MERS-MA30-6-1-2.

With the aim of testing possible changes in the virulence of MERS-MA30 i.e. the recombinant virus obtained in the present invention by chemical synthesis, incorporating the mutations acquired by a MERS-CoV when passed 30 times in knockin (KI) mice, an inoculation was performed in humanised KI mice with the passaging-derived virus (MERS-MA30-6-1-2), which best reproduces in the mouse the clinical signs observed in humans (Li et al., 2017), and with the chemically synthesised and genetically engineered virus (MERS-MA30). Mice were intranasally inoculated with 1×10⁵ PFU/mouse. It was observed that weight losses and survival were similar with the two viruses, indicating that the virus recovered from the engineered infectious cDNA (MERS-MA30) behaved virtually the same as the isolated virus (MERS-MA30-6-1-2), with slightly, but not significantly, higher virulence (FIG. 5).

3. Construction of RNA Replicons from the Recombinant MERS-MA30 Infectious Clone

The specific data for each of the genes, present in the pBAC, encoding MERS-CoV are shown in Table 1.

The pBAC-MERS-MA30-FL was used as the basis for engineering the different MERS-MA30 replicons and mutant viruses (FIG. 3):
MERS-MA30 (full-length starting cDNA),
MERS-MA30-ΔE RNA replicon (deletion of the gene encoding E protein),
MERS-MA30-Δ5-ΔE RNA replicon (deletion of genes 5 and E),
MERS-MA30-Δ [3-5] mutant virus (deletion of ORFs 3, 4a, 4b and 5), and
MERS-MA30-Δ RNA replicon [3-E] (deletion of ORFs 3, 4a, 4b, 5 and E).

For the generation of the infectious cDNA of MERS-MA30-ΔE and MERS-MA30-Δ5-ΔE, a 502-bp chemically synthesised fragment (GeneArt, Thermo Fisher Scientific) flanked by KfII/SanD1 and Pfl23II restriction sites was designed. This fragment included the MERS-MA30 mutations between nucleotides 27535 and 28236 of the viral genome, the CS deletion of the transcription regulating sequence (TRS) of the gene encoding E protein, and the deletion of the first 197 nucleotides of the sequence of the gene encoding E protein. The designed fragment is shorter in length as it does not comprise the entire E gene sequence. This fragment was inserted at position 27535 and 28236 as there is one restriction site for KfII/SandD1 (see FIG. 2B) and the other restriction site Pfl23II is originally located into FS-9, which was introduced in pBAC-SA-F6.

The last 52 nucleotides of the sequence of the gene encoding E protein were maintained as they include part of the TRS of M gene. The synthesised fragment was cloned into the intermediate plasmid pBAC-SA-F6 (positions 25841 to 30162 of the viral genome) to generate a pBAC-SA-F6-MA30-ΔE, and into pBAC-SA-F6-MA30-Δ5 to generate a pBAC-SA-F6-MA30-Δ5-ΔE. The PacI-RsrII fragment of pBAC-SA-F6-MA30-ΔE and pBAC-SA-F6-MA30-Δ5-ΔE, which includes the KfII-Pfl23II region, was cloned into pBAC-MERS-MA30-FL to obtain the corresponding pBAC-MERS-MA30-ΔE and pBAC-MERS-MA30-Δ5-ΔE. Digestions of vectors with restriction enzymes were performed according to the manufacturer's instructions.

For the construction of MERS-MA30-Δ[3,4a,4b,5] and MERS-MA30-Δ[3,4a,4b,5,E] infectious cDNAs, an intermediate plasmid pUC57-F5-Δ3-MERS-MA30 was previously generated from pUC57-F5-Δ3-MERS (Almazan et al., 2013). The pUC57-F5-Δ3-MERS-MA30 includes the mutations acquired by MERS-MA30-6-1-2 in the region of the viral genome between nucleotides 20902 and 25840, as well as the deletion of the ORF3 gene. This region, flanked by SwaI and PacI restriction sites, was cloned into pBAC-MERS-MA30-FL and pBAC-MERS-MA30-ΔE to obtain a pBAC-MERS-MA30-Δ3 and a pBAC-MERS-MA30-Δ3-ΔE, respectively. Finally, a digestion with PacI and KfII/SanD1 was performed to delete genes 4a, 4b and 5, the fragments were separated by agarose gel electrophoresis and the digested vectors were purified. Since the ends resulting from the digestion were not cohesive with each other, blunt ends were generated with T4 phage DNA polymerase (New England Biolabs). For this, 300 ng of each digested plasmid were incubated with 1 U of enzyme per 1 μg of DNA for 30 minutes at 37° C. in the presence of dNTPs excess. The enzyme was then inactivated by treatment at 75° C. for 20 minutes and T4 phage DNA ligase (Roche) was added to ligate the ends, generating plasmids pBAC-MERS-MA30-Δ[3-5] and pBAC-MERS-MA30-Δ[3,4a,4b,5,E].

Optionally this replicon may comprise the polynucleotide sequence of the gene encoding S protein optimised for expression in mammalian cells by the procedure described in the next section.

4. Obtaining V1-CD and V1-VLP RNA Replicons

To generate the V1-CD and V1-VLP replicons, two 4945 bp fragments, flanked by SwaI and PacI restriction sites, were chemically synthesised (GeneArt, Thermo Fisher Scientific) (FIG. 2B). These fragments, named Sopt-CD and Sopt-VLP contained nucleotides 20898 to 25844 of the MERS-MA30 genome, in which the sequence of the gene encoding S protein (Table 1, SEQ_ID 4) was optimised for expression in humans using an online codon optimisation tool (https://en.vectorbuilder.com/tool/codon-optimization.html). In this process, we avoided creating new restriction sites from among those used for MERS-CoV cDNA assembly (FIG. 2B) (Almazan et al, 2013). Thus, the Sopt-VLP fragment contained SEQ_ID 3. In the case of the Sopt-CD fragment, sequence optimisation also took into account that no T7 polymerase termination sites, such as ATCTGTT, were generated, and nucleotide changes resulting in the amino acid substitutions V1060P and L1061P were included. Thus, the Sopt-CD fragment contained the SEQ_ID 7 sequence. The Sopt-CD and Sopt-VLP fragments were digested with SwaI and PacI and cloned into the same sites of the pBAC-MERS-MA30-Δ3-ΔE plasmid. Finally, a digestion with PacI and KfII/SanDI was performed to delete genes 4a, 4b and 5, the fragments were separated by agarose gel electrophoresis and the digested vectors were purified. Since the ends resulting from the digestion were not cohesive with each other, blunt ends were generated with T4 phage DNA polymerase (New England Biolabs). For this, 300 ng of each digested plasmid was incubated with 1 U of enzyme per 1 μg of DNA for 30 minutes at 37° C. in the presence of dNTPs excess. The enzyme was then inactivated by treatment at 75° C. for 20 min and T4 phage DNA ligase (Roche) was added to ligate the ends, generating plasmids pBAC-MERS-MA30-Sopt-CD-Δ[3, 4a, 4b, 5, E] and pBAC-MERS-MA30-Sopt-VLP-Δ[3,4a,4b,5,E], which are the basis for the V1-CD and V1-VLP replicons, respectively.

To include the nsp1-ΔD deletion, two fragments flanked by AscI and BbvCI restriction sites were chemically synthesised (GeneArt, Thermo Fisher Scientific): T7-nsp1-ΔD and nsp1-ΔD. These fragments contained the T7 promoter (T7P) or CMV promoter, respectively, and nucleotides 1 to 3123 of the MERS-MA30 genome, plus the deletion of nucleotides 792 to 827 of the MERS-MA30 genome. These fragments were digested with AscI and BbvCI enzymes and cloned into the same sites of the plasmids pBAC-MERS-MA30-Sopt-CD-Δ[3,4a,4b,5,E], for T7-nsp1-ΔD, and pBAC-MERS-MA30-Sopt-VLP-Δ[3,4a,4b,5,E], for nsp1-ΔD, leading to plasmids pBAC-MERS-MA-V1-CD and pBAC-MERS-MA-V1-VLP, respectively. These plasmids contained the sequences of the V1-CD and V1-VLP replicons (FIG. 4, SEQ_ID 9 and SEQ_ID 11).

To include the nsp1-ΔC deletion, two fragments flanked by AscI and BbvCI restriction sites were generated by chemical synthesis (GeneArt, Thermo Fisher Scientific): T7-nsp1-ΔC and nsp1-ΔC. These fragments contained the T7 promoter (T7P) or CMV promoter, respectively, and nucleotides 1 to 3123 of the MERS-MA30 genome, plus the deletion of nucleotides 708 to 734 of the MERS-MA30 genome.

To include a deletion in the nsp1 gene, two fragments flanked by the restriction sites AscI and BbvCI: T7-nsp1-Δ and nsp1-Δ were generated by chemical synthesis (GeneArt, Thermo Fisher Scientific). These fragments contained the T7 promoter (T7P) or CMV, respectively, and nucleotides 1 to 3123 of the MERS-MA30 genome, plus a deletion of between 27 and 36 nucleotides between positions 528 and 848 of the MERS-MA30 genome.

The above deletions in the gene encoding the nspl protein can be combined in any way and are included in the same fragment containing the T7 or CMV promoter.

5. In Vitro Transcription of the V1-CD Replicon RNA

A previously described protocol for in vitro transcription of coronavirus RNAs (Eriksson K. K. et al, 2008, Methods in Mol. Biol. 454:237-254) was followed, with minor modifications. All processes were performed under RNase-free conditions and with RNase-free reagents. In the transcription reaction, 1 μg of undigested pBAC-MERS-MA-V1-CD plasmid as template, a Ribo m⁷G cap analogue (Promega) and the RiboMAX Large Scale RNA production system kit (Promega) were used. The final volume of each reaction was 50 μl, which were incubated for 2 h at 30° C. Subsequently, the template DNA was removed by adding 2 μl of RNase-free DNase and incubating the mixture at 37° C. for 20 min. Finally, the RNA was precipitated with LiCl, resuspended in 30 μl of RNase-free water and stored at −80° C. until use.

For administration into cell cultures, Lipofectamine 2000 (Life Technologies) was used under the same conditions as those used to transfect DNA. For administration to mice, the in vivo-jetRNA reagent (Polyplus transfection) was used, following the manufacturer's recommendations.

6. Replication capacity or amplification of MERS-MA30-derived replicons in the presence or absence of the E protein. The replicative capacity of the constructed mutants was assessed in Huh-7 cells. These cells had previously been transiently transfected with the inducible plasmid TRE-Auto-rtTA-V10-2T-E-MERS-CoV expressing the E protein. The growth medium contained doxycycline at a concentration of 1000 ng/mL for induction of E protein expression. At 5 hpt, cells were infected with virus at an MOI (multiplicity of infection) of 0.001 and infection was monitored for 72 hours.

Other methods have been developed for the generation of packaging cell lines based on E protein mutants that inactivate E protein toxicity and provide highly efficient packaged MERS-CoV-derived RNA replicons.

In cells grown in medium without doxycycline, i.e. in the absence of the externally added E protein, MERS-MA30, and MERS-MA30-Δ[3,4a,4b,5] viruses followed very similar growth kinetics, with no significant differences (FIG. 6). In the growth of the MERS-MA30-Δ[3,4a,4b,5] mutant, a reduction in titer was observed at 24 hpi compared to that of the MERS-MA30 virus, possibly as a consequence of the absence of the accessory genes. The MERS-MA30-ΔE and MERS-MA30-Δ [5,E] replicons behaved similarly to the MERS-CoV-ΔE replicon, which does not propagate in the absence of the gene encoding the E protein (data not shown). Finally, a significant decrease in the growth of the MERS-MA30-Δ[3,4a,4b,5,E] replicon was observed at 24 and 48 hpi compared to the growth of the other replicons (MERS-MA30-ΔE and MERS-MA30-Δ[5,E]), suggesting that the joint deletion of genes 3, 4a, 4b, 5 and E had a greater effect than the deletion of the gene encoding the E protein alone, or the joint deletion of genes 5 and E (FIG. 6).

No major differences were observed in the titers of the MERS-MA30-Δ[3,4a,4b,5] mutant, which only increased 2- to 4-fold in the presence of the E protein, and remained below the MERS-MA30 virus titers (FIG. 6). However, the titers of the MERS-MA30-ΔE and MERS-MA30-Δ[5,E] replicons increased in the presence of the E protein, reaching levels similar to those of MERS-MA30 at 72 hpi. Despite the availability of the E protein, the MERS-MA30-Δ[3,4a,4b,5,E] replicon showed slower growth than the MERS-MA30-ΔE and MERS-MA30-Δ[5,E] replicons. However, late in infection, it reached similar titers to the MERS-MA30 viruses in the presence of the E protein.

Complementation with the E protein in trans allows replicons with the gene encoding the E protein deleted to achieve at late times similar titers to those of the viruses from which they are derived. Moreover, deletion of accessory genes 3, 4a, 4b, and 5 resulted in attenuated growth of the MERS-MA30-Δ[3,4a,4b,5] mutant and the MERS-MA30-Δ[3,4a,4b,5,E] replicon, compared to the MERS-MA30 virus and the MERS-MA30-ΔE replicon, respectively, which included these genes. The absence of gene 5 did not appear to affect the growth of MERS-CoV in cell culture in the case of the MERS-MA30-Δ[5,E] replicon.

7. Analysis of Virion Production by MERS-CoV-ΔE Replicon Compared with MERS-CoV WT Virus.

A comparative study of the morphogenesis of MERS-CoV WT virus and MERS-CoV-ΔE replicon was carried out. For this purpose, Huh-7 cells were infected in the absence of E protein. At 17 hpi, cells were embedded in resin, and sections were taken for observation by transmission electron microscopy (TEM) (FIG. 7).

MERS-CoV-ΔE and MERS-MA30-Δ[3,4a,4b,5,E] formed virions inside the cell that were apparently similar to those formed by MERS-CoV WT, although only the VLPs formed by MERS-CoV-ΔE are shown. In cells infected with an MOI 1, MERS-CoV VVT showed a high cytopathic effect, with virus vesicles filled with spherical-shaped virions, whereas MERS-CoV-ΔE replicon vesicles were less frequent, with elongated shapes and fewer immature virions. A similar pattern was observed in cells infected with a MOI of 0.1 (results not shown), although the cytopathic effect in MERS-CoV WT infection was lower compared to infection at MOI 1. These results demonstrated that the MERS-CoV-ΔE replicon formed polymeric structures with high immunogenic potential.

8. Attenuation of MERS-MA30 Mutants in KI Mice.

The pathogenicity of MERS-MA30-Δ[3,4a,4b,5] virus and MERS-MA30-ΔE, MERS-MA30-Δ[5,E] and MERS-MA30-Δ[3,4a,4b,5,E] replicons was evaluated in 16-week-old KI mice (Li et al., 2017). MERS-MA30 was used as a virulent reference virus. Of each virus or replicon, 1×10⁴ PFU were intranasally inoculated, and weight loss and survival were monitored for the next 13 days (FIG. 8).

All mice inoculated with MERS-MA30 virus lost weight and died between 6 and 8 dpi. In contrast, mice infected with MERS-MA30-Δ[3,4a,4b,5] or with MERS-MA30-ΔE, MERS-MA30-Δ[5,E] or MERS-MA30-Δ[3,4a,4b,5,E] replicons did not lose weight, and all of them survived, indicating that all generated deletion mutants were attenuated. Lungs of mice inoculated with MERS-MA30 and MERS-MA30-Δ[3,4a,4b,5,E] were sampled at 3 and 6 dpi. From these samples, viral titer (FIG. 9), replication and transcription were analysed (FIG. 10). While high titers were detected in the lungs of MERS-MA30-infected mice at 3 dpi that decreased at 6 dpi, no virus was observed in the lungs of MERS-MA30-Δ[3,4a,4b,5,E] replicon-infected mice by immunofluorescence focus formation detection assay (FIG. 9). This result was consistent with previous in vitro results showing that in the absence of the gene encoding E protein, MERS-CoV does not spread. Also, replication and transcription levels of the MERS-MA30-Δ[3,4a,4b,5,E] replicon were significantly lower than those of the MERS-MA30 virus (FIG. 10), since the MERS-MA30-Δ[3,4a,4b,5,E] replicon does not spread to other cells in vivo and only replicates in those cells initially infected.

9. Protection Provided by MERS-MA30 Deletion Mutants in KI Mice.

Mice immunised with the different deletion mutants were challenged 21 days post-immunisation (dpim) with a lethal dose of MERS-MA30 (1×10⁵ PFU per mouse) (FIG. 11). Non-immunised control mice lost weight and died between 6 and 7 dpi. However, all mice immunised with one of the deletion mutants survived the challenge, and none of them suffered significant weight loss.

During the challenge, samples were taken at 2, 4 and 6 days post-challenge (dpc) from the lungs of challenged mice to analyse the protection conferred by MERS-MA30-Δ[3,4a,4b,5,E], and were used to measure viral replication and transcription, and virus titer. In the lungs of non-immunised mice, elevated replication and transcription levels were detected, which decreased slightly more than tenfold at days 4 and 6, but were still significantly high (FIG. 12). In contrast, replication and transcription levels in the lungs of mice immunised with the MERS-MA30-Δ[3,4a,4b,5,E] replicon were significantly lower at all post-challenge times tested (FIG. 12). Interestingly, no challenge virus growth was detected in the lungs of immunised mice at any time post-challenge (2, 4 and 6 dpc) (FIG. 13), so it can be argued that MERS-MA30-Δ[3,4a,4b,5,E] confers sterilising immunity, i.e. it does not allow the virus to grow after immunisation.

Neutralising antibody levels were determined in serum from mice immunised with the MERS-MA30-Δ[3,4a,4b,5,E] replicon and control mice (without the replicon) at 0 and 21 dpim by a neutralisation assay. Antibody titers are expressed as the highest dilution showing complete neutralisation of the cytopathic effect in 50% of the wells (TCID₅₀) (FIG. 14). No neutralising antibodies were detected in the serum of non-immunised mice or mice immunised with the MERS-MA30-Δ[3,4a,4b,5,E] replicon at 0 dpim. However, at 21 dpim, mice immunised with the MERS-MA30-Δ[3,4a,4b,5,E] replicon showed detectable levels of neutralising antibodies compared to non-immunised mice after a single immunisation.

Taken together, these results demonstrated that virus MERS-MA30-Δ[3,4a,4b,5] and replicons MERS-MA30-ΔE, MERS-MA30-Δ[5, E] and MERS-MA30-Δ[3,4a,4b,5, E] induced protection in the experimental KI mouse model against a lethal dose of MERS-MA30 virus, and that the MERS-MA30-Δ[3,4a,4b,5,E]-derived RNA replicon is a safe, effective and very promising vaccine candidate. Similar results were obtained with the humanised transgenic mouse model (K18) and the replicon with the human virus sequence, not adapted to mice.

### LIST OF SEQUENCES

This specification comprises the following sequences:

SEQ_ID 1: Nucleotide sequence of the vector containing the complete pBAC-MERS-CoV-Δ[3,4a,4b,5,E] replicon. Includes pBAC sequence (nucleotides 1 to 7889), RNA replicon (nucleotides 7890 to 35838) and pBAC sequence (nucleotides 35839 to 36179)

SEQ_ID 2: Nucleotide sequence of the vector containing the full-length pBAC-MERS-MA30-Δ[3,4a,4b,5,E] replicon. Includes pBAC sequence (nucleotides 1 to 7889), RNA replicon (nucleotides 7890 to 35838) and pBAC sequence (nucleotides 35832 to 36173)

SEQ_ID 3: Nucleotide sequence of the gene encoding the S protein of MERS-MA30-CoV with codons optimised for expression in mammalian cells

SEQ_ID 4: Nucleotide sequence of the gene encoding MERS-MA30-CoV S protein with codons not optimised for expression in mammalian cells

SEQ_ID 5: Nucleotide sequence of the gene encoding MERS-CoV protein S with codons optimised for expression in mammalian cells

SEQ_ID 6: Nucleotide sequence of the gene encoding MERS-CoV Protein S with codons not optimised for expression in mammalian cells

SEQ_ID 7: Nucleotide sequence of the gene encoding MERS-MA30-CoV protein S with codons optimised for expression in mammalian cells and modifications 24633_24634 delins CC and 24637_24638 delins CC

SEQ_ID 8: Nucleotide sequence of the gene encoding the S protein of MERS-CoV with codons optimised for expression in mammalian cells and modifications 24633_24634 delins CC and 24637_24638 delins CC

SEQ_ID 9: Nucleotide sequence of the chemically defined MERS-MA30-V1-CD replicon

SEQ_ID 10: Nucleotide sequence of the chemically defined MERS-CoV-V1-CD replicon

SEQ_ID 11: Nucleotide sequence of the MERS-MA30-V1-VLP replicon

SEQ_ID 12: Nucleotide sequence of the MERS-CoV-V1-VLP replicon

SEQ_ID 13: Nucleotide sequence of the T7P promoter

SEQ_ID 14: Nucleotide sequence of the 5′UTR

SEQ_ID 15: MISC nucleotide sequence

SEQ_ID 16: DLP nucleotide sequence

SEQ_ID 17: P2A nucleotide sequence

SEQ_ID 18: 3′UTR nucleotide sequence

SEQ_ID 19: PolyA tail nucleotide sequence

SEQ_ID 20: T7 terminator nucleotide sequence

SEQ_ID 21: Nucleotide sequence of MERS-MA30 virus

SEQ_ID 22: Primer nucleotide sequence (PCR primer) of VS-EcoRI-E-MERS-rtTA-V10-2T

SEQ_ID 23: Primer nucleotide sequence (PCR primer) of RS-EcoRI-EMERS-rtTA-V10-2T

SEQ_ID 24: Primer nucleotide sequence (PCR primer) of RS-E-MERS

SEQ_ID 25: Primer nucleotide sequence (PCR primer) of VS-TRE-Auto-2380

SEQ_ID 26: Primer nucleotide sequence (Taqman probes) gRNA-MERS

SEQ_ID 27: Primer nucleotide sequence (Taqman probes) sgmRNA-N-MERS

SEQ_ID 28: Primer nucleotide sequence (PCR primer) of VS MERS gRNA

SEQ_ID 29: Primer nucleotide sequence (PCR primer) of RS MERS gRNA

SEQ_ID 30: Primer nucleotide sequence (PCR primer) of Leader sgRNA

SEQ_ID 31: Primer nucleotide sequence (PCR primer) of sgRNA-N

### REFERENCES

Almazán, F., DeDiego, M. L., Sola, I., Zuniga, S., Nieto-Torres, J. L., Marquez-Jurado, S., Andres, G., Enjuanes, L., 2013. Engineering a replication-competent, propagation-defective Middle East respiratory syndrome coronavirus as a vaccine candidate. MBio 4, e00650-00613.

Almazán, F., González, J. M., Penzes, Z., Izeta, A., Calvo, E., Plana-Duran, J., Enjuanes, L., 2000. Engineering the largest RNA virus genome as an infectious bacterial artificial chromosome. Proc. Natl. Acad. Sci. USA 97, 5516-5521.

Almazán, F., Marquez-Jurado, S., Nogales, A., Enjuanes, L., 2015. Engineering infectious cDNAs of coronavirus as bacterial artificial chromosomes. Methods Mol. Biol. 1282, 135-152.

Almazán, F., Sola, I., Zuñiga, S., Marquez-Jurado, S., Morales, L., Becares, M., Enjuanes, L., 2014. Coronavirus reverse genetic systems: Infectious clones and replicons. Virus Res. 189, 262-270.

Das, A. T., Tenenbaum, L., Berkhout, B., 2016a. Tet-On Systems For Doxycycline-inducible Gene Expression. Curr. Gene Ther. 16, 156-167.

Das, A. T., Zhou, X., Metz, S. W., Vink, M. A., Berkhout, B., 2016b. Selecting the optimal Tet-On system for doxycycline-inducible gene expression in transiently transfected and stably transduced mammalian cells. Biotechnol. J. 11, 71-79.

González, J. M., Penzes, Z., Almazán, F., Calvo, E., Enjuanes, L., 2002. Stabilization of a full-length infectious cDNA clone of transmissible gastroenteritis coronavirus by insertion of an intron. J. Virol. 76, 4655-4661.

Jimenez-Guardeño J M, Regla-Nava J A, Nieto-Torres J L, DeDiego M L, Castaño-Rodriguez C, Fernandez-Delgado R, Perlman S, Enjuanes L. Identification of the Mechanisms Causing Reversion to Virulence in an Attenuated SARS-CoV for the Design of a Genetically Stable Vaccine. PLoS Pathog. 2015 Oct. 29; 11(10):e1005215. doi: 10.1371/journal.ppat.1005215. PMID: 26513244; PMCID: PMC4626112.

Li, K., Wohlford-Lenane, C. L., Channappanavar, R., Park, J. E., Earnest, J. T., Bair, T. B., Bates, A. M., Brogden, K. A., Flaherty, H. A., Gallagher, T., Meyerholz, D. K., Perlman, S., McCray, P. B., Jr., 2017. Mouse-adapted MERS coronavirus causes lethal lung disease in human DPP4 knockin mice. Proc. Natl. Acad. Sci. USA 114, E3119-E3128.

Livak, K. J., Schmittgen, T. D., 2001. Analysis of relative gene expression data using real-time quantitative PCR and the 2(-Delta Delta C(T)) Method. Methods 25, 402-408.

Lundstrom, K., 2018. Self-replicating RNA viruses for RNA therapeutics. Molecules 23, 3310.

Mairhofer, J., Grabherr, R., 2008. Rational vector design for efficient non-viral gene delivery: challenges facing the use of plasmid DNA. Mol. Biotechnol. 39, 97-104.

Mairhofer, J., Pfaffenzeller, I., Merz, D., Grabherr, R., 2008. A novel antibiotic free plasmid selection system: advances in safe and efficient DNA therapy. Biotechnol. J. 3, 83-89.

Ortego, J., Escors, D., Laude, H., Enjuanes, L., 2002. Generation of a replication-competent, propagation-deficient virus vector based on the transmissible gastroenteritis coronavirus genome. J. Virol. 76, 11518-11529.

Pallesen J, Wang N, Corbett K S, et al. Immunogenicity and structures of a rationally designed prefusion MERS-CoV spike antigen. Proc Natl Acad Sci USA. 2017; 114(35):E7348-E7357. doi:10.1073/pnas.1707304114

Reed, L. J., Muench, H., 1938. Virus titers (50% tissue culture infectious doses/ml, TCID50/ml) were calculated using the method of Reed & Muench. Am. J. Hyg. 27, 493-497.

Sambrook, J., Russell, D. W., 2001. Molecular cloning: A laboratory manual, 3rd ed. Cold Spring Harbor Laboratory Press, Cold Spring Harbor, N.Y.

van Boheemen, S., de Graaf, M., Lauber, C., Bestebroer, T. M., Raj, V. S., Zaki, A. M., Osterhaus, A. D., Haagmans, B. L., Gorbalenya, A. E., Snijder, E. J., Fouchier, R. A., 2012. Genomic characterization of a newly discovered coronavirus associated with acute respiratory distress syndrome in humans. MBio 3, e00473-00412.

Wang, K., Boysen, C., Shizuya, H., Simon, M. I., Hood, L., 1997. Complete nucleotide sequence of two generations of a bacterial artificial chromosome cloning vector. Biotechniques 23, 992-994.

1. An RNA replicon derived from a coronavirus to which it has been deleted comprising:
partially the gene encoding the E protein retaining the last 52 nucleotides of the sequence of this gene and
total or partially at least 4 genes encoding genus accessory proteins selected from 3, 4a, 4b and 5 of MERS CoV retaining 39 nucleotides of the sequence of the gene encoding genus accessory protein 3, said 39 nucleotides are located in position 33409-33447 of SEQ_ID 1, and
an identity of at least 95% or 96% or 97% or 98% or 99% with respect to the sequence SEQ_ID 1, namely with respect to the fragment comprised from nucleotides 7890 to 35838.

2.-8. (canceled)

9. The RNA replicon according to claim 1, having a size between 18 and 29 kb.

10.-11. (canceled)

12. The RNA replicon according to claim 1, wherein the RNA replicon is wrapped within a VLP-E+, comprising E protein provided in trans.

13.-14. (canceled)

15. A method for preparing an RNA replicon defined in claim 1 comprising:
constructing the full-length cDNA from the gRNA of a coronavirus and inserting it into an expression vector obtaining an infectious clone;
partially deleting the gene encoding the E protein; and
totally or partially deleting of at least 4 genes encoding genus accessory proteins selected among 3, 4a, 4b and 5 of MERS-CoV; and
transfecting the upstream expression vector into a host cell under conditions suitable for its expression.

16. (canceled)

17. The method according to claim 15, comprising total or partial deletion of the gene encoding E protein retaining the last 52 nucleotides of the sequence of this gene and genes encoding genus accessory proteins of 3, 4a, 4b and 5 of MERS-CoV retaining 39 nucleotides of the sequence of the gene encoding genus accessory protein 3, said 39 nucleotides are located in position 33409-33447 of SEQ_ID 1.

18. The method according to claim 15, wherein the full-length cDNA is obtained by chemically synthesizing several fragments and introducing said fragments into an expression vector.

19. The method according to claim 15, wherein the total or partial deletion of genes from the genome is selected between: use of restriction enzymes, vectors recombination and CRISPR technology.

20. (canceled)

21. An expression vector comprising the cDNA sequence complementary to the RNA replicon defined in claim 1.

22. (canceled)

23. The expression vector according to claim 21, which is selected from a bacterial artificial chromosome, a cosmid and a P1-derived artificial chromosome.

24. The expression vector according to claim 23, comprising a selection system for cells carrying said vector selected from the group of:
an antibiotic resistance gene, preferably chloramphenicol, kanamycin or neomycin,
a selection system based on the complementation of auxotrophic markers, preferably the DapD or tpiA gene
a toxin/antitoxin mechanism, preferably, the hok/sok system or ccdB/ccdA,
a ColE1-based repression mechanism, and
a mechanism based on the counter-selection marker sacB.

25. A vaccine composition capable of inducing protection in a subject, against infection caused by a coronavirus, such that said vaccine composition, comprising an RNA replicon according to claim 1, together with, optionally:
at least one pharmaceutically acceptable excipient and/or.
at least one chemical or biological adjuvant or immunostimulant.

26. The vaccine composition according to claim 25, for its administration to a subject topically, intranasally, orally, subcutaneously or intramuscularly.

27. The vaccine composition according to claim 26, for its administration to the subject simultaneously together with a chemical or biological adjuvant or immunostimulator.

28. The vaccine composition according to claim 25, for its administration to the subject before or after the chemical or biological adjuvant or immunostimulator.

29. The vaccine composition according to claim 25, which is in liquid or lyophilized form.

30. The vaccine composition according to claim 25, wherein the subject is a mammal, selected from a human or a domestic animal selected from a dog, and/or a cat.

31. Method of use of the RNA replicon defined in claim 1 as a vaccine composition comprising its administration to a subject topically, intranasally, orally, subcutaneously or intramuscularly.

32. The RNA replicon according to claim 1, having a size between 20 and 27 kb.

33. The RNA replicon according to claim 1, having a size between 22 and 26 kb.

34. The RNA replicon according to claim 1, having a size between 22 and 24 kb.
